(19) United States
(12) Patent Application Publication
(10) Pub. No.: US 2011/0171663 A1
(43) Pub. Date: Jul. 14, 2011

(54) MICROTRENCH AND TUMOUR PROLIFERATION ASSAY

(75) Inventors: Paul J. Smith, Cardiff (GB); Rachel J. Errington, Cardiff (GB); Huw Summers, Swansea (GB); Sally Chappell, Cardiff (GB)

(73) Assignee: University College Cardiff Consultants Ltd, Cardiff (GB)

(21) Appl. No.: 12/865,181

(22) PCT Filed: Jan. 29, 2009
(86) PCT No.: PCT/GB09/00244

§ 371 Date: Oct. 4, 2010

(30) Foreign Application Priority Data
Jan. 29, 2008 (GB) 0801600.8

(51) Int. Cl. G01N 33/50; C12Q 1/02; C12M 3/00; C40B 40/02; C12N 5/09

(52) U.S. Cl. 435/7.21; 435/29; 435/289.1; 506/14; 435/325

(57) ABSTRACT

There is provided a cell culture microtrench being defined on or in a surface of a substrate, wherein the ratio of the width of the microtrench to the maximum length of the short axis of a cell type of interest is about 6 or preferably less, the length of the short axis of the cell type being measured when a cell is in detached or suspended form. There is also provided an array comprising such a microtrench and uses of such microtrenches, including cell-based assays.

### FIELD OF THE INVENTION

The invention relates to the use of a cell culture microtrench having dimensions which allow attachment of cells to the surface of the trench and constrain the location and behaviour of cells for analytical advantage. The invention also relates to such a microtrench, arrays of such trenches with varying dimensions to provide for selection of optimal assay characteristics for a user-defined purpose, and arrays of trenches with the same dimensions to facilitate the scaling up and efficiency of a given assay. The trenches and/or arrays can be readily incorporated into convenient methods of cellular analysis in research, diagnostics and screening assays.

### BACKGROUND

### Confounding Factors in Cell-Based Assays

The determination of the abilities of cultured cells to divide, grow, survive and display molecular features or behavioural characteristics underpin many biological analyses in basic cellular research, the evaluation of cell therapeutic preparations, biotechnology processes, diagnostic studies and in the screening of drugs and biopharmaceuticals. There exists a need to make biological assays faster and simpler to perform, with an overriding drive to make the processes cheaper, yet maintain accuracy and reproducibility. This is due to a rapid increase in the number of research and diagnostic assays. In research and the drug discovery process, cell-based assays allow for functional measurements of complex behaviour, whilst retaining the relevance of living systems.

In vitro assays and methods are often constrained by the characteristics of the cell type under study, including the preference to proliferate in an adherent form attached to a supporting substrate overlaid by a nutrient medium, or in non-adherent forms such as single-cell suspensions or multicellular aggregates. One approach has been to enable growth in a semi-solid support medium to constrain cell movement, thereby providing an opportunity to study clonal clusters for non-adherent cells or, indeed, adherent cells capable of adaptation to growth in semi-solid support medium. A resultant problem is that the cell is in a 3-D cluster in these circumstances, making individual cell analysis difficult, fixation problematic and divisional history effectively impossible to determine. Adherent cultures offer the convenient advantage of imposing a degree of restriction to the three-dimensional position of a single cell facilitating direct observations with time and the application of imaging solutions for cell analyses. Adherent cell assays are carried out across a range of instrumentation, frequently comprising imaging or other means of optically sensing cells on a biocompatible surface. Adherence or cell attachment potential is both a quality of the cell and of the substrate itself. Typically, assays involving adherent cells seek to enhance cell attachment/adherence to the cell surface so that cell detachment and loss are reduced.

Increasingly, there is a need for micro-scaled cell-based assays, but these must contend with the confounding factors presented by the natural behaviour of cells, given their frequent need for substrate interaction to effect survival, proliferation and motility. These confounding factors contribute to the ‘degrees of freedom’, within a given analytical process, imposed by the differential expression of cell-associated characteristics that act against the application of rapid, definitive and high throughput approaches. Such factors, each of which has an impact on the accuracy and/or ease of use of adherent cell-based assays, include:

Cell proliferation—this is a fundamental property of a cellular population and is reflected by the increase in cell number over time. A cell population can contain a range of proliferation components (i.e., not all cells behave the same) causing problems for identifying and determining the proportion of cells that have not undergone cell division as distinct from those that have undergone rounds of division to produce cell lineages, particularly if the division event has not been visually ‘captured’ during the analysis. Further, inherent or induced differences in proliferation rates between cells can impose asynchrony, demanding kinetic analyses of single cells.

Cell motility—this is a fundamental behavioural characteristic which varies across cell types and is affected by culture conditions and is represented as cell movement across a substrate. Motility can cause a problem for cell re-identification when re-visiting the original location of a cell at a later time point and furthermore potential removal of a cell from the enumerated fraction (see also cell incursion). Motility necessarily causes changes in cell morphology.

Cell loss—this causes a problem for enumeration of a fraction of cells that may be lost (e.g., as the result of cell death) in mixed populations which are also undergoing cell proliferation. If cell loss is restricted to a fraction of the population, then its underlying extent may be misjudged due to the proliferation of surviving fractions.

Cell incursion—relating to the adventitious appearance of cells from outside a field undergoing analysis/observation (e.g., via cell motility or a process of detachment and reattachment) effectively resulting in a ‘contamination’ of a field of interest increasing the noise in the system.

Cell morphology—changes in morphology occur in the normal life cycle of a cell and this is, therefore, a highly informative assay output. This can cause problems for cell identification particularly when applying image analysis or segmentation algorithms, whereby cell shape changes may act to confound analysis procedures or reduce their efficiency.

Cell-to-cell heterogeneity—the fundamental concept that not one cell in a population is in the same state as the next in a cell-based assay (i.e., cell-to-cell heterogeneity) can be a confounding factor or, alternatively, a feature of interest.

Microniche exhaustion of growth and survival factors—when cells are constrained within diffusion-limited circumstances (e.g., in a micropocket), there is the potential to limit access to vital molecules in the supporting medium and access to analytical reagents. Further, separation of cells into separated micro-niche areas effectively reduces local cell density and may thereby reduce the ability of cell cultures to ‘condition’ to their overall environment.

Low event frequency—cellular events of interest for analytical process (e.g., a transition of a cell through mitosis or the episodic appearance of a reporting molecule) may be relatively rare and asynchronous within a cell population. To detect sufficient events for the purposes of an assay requires windows of observation that can capture the event together with a large enough population to present events. The common solution is to use high density cultures to enhance the packing density for events of interest. This approach leads to further complications with respect to increased cell interactions that can compromise assay performance. Another common solution is to increase the number of observed views of a population although this leads to an increase in the amount of data collected, which must then be stored.

Cell-cell interaction—it is recognised that cellular interactions can modify cellular behaviour in vitro (e.g., through contact inhibition of proliferation in primary fibroblast cultures or the formation of cell-cell gap junction communication synapses). Increasing cellular density in an attempt to improve the efficiency, detection limits or the relative signal-to-noise ratio within an assay will modify opportunities for cell-cell interaction and introduce possible sources of biological variation.

Cell lineages and inter-relationships—as a cell lineage forms from an original progenitor there is an increasingly complex set of relationships between cells that demands individual cell tracking to establish. Uncertainty in terms of progenitor identification and descendant relationships introduces noise into an analytical system. The term progenitor as used herein refers to a cell which is nominated as an originator of a line of descent, i.e., a founder of a lineage of cells.

Cellular substrate-attachment and substrate-adherence potential—it is recognised that cells vary in their capacity to interact with and become adherent to a given substrate according to cell-associated characteristics of type, behaviour and state.

### Micropatterning of Adherence Substrates to Control Aspects of Cell Behaviour

Some of the above degrees of freedom, pertaining to an adherent cell-based assay, can be influenced by the nature of the substrate including its microtopochemistry and microtopographical characteristics. One concern is that the active ‘patterning’ of an adherence substrate should allow cell cultures to be conveniently analysed using optical microscopy (Joos et al. (2006) Eur J. Cell Biol. 85 225-8). Microtopochemistry and microtopographical modulation of a surface has been reported for the control of the deposition and orientation of cells using a range of methods (Falconnet et al. (2006) Biomaterials 27 3044-63). Such control is advantageous both in some aspects of research and in the development of cell-based assays in which the location of a cell or cell cluster can be defined. For example a recently published patent application (Rosenthal et al., US2007/0238089-A1) describes the use of a patterned microwell device.

Microfeatured surfaces have been produced that are within the scale range for human cells, although it is recognised that cell size can vary in a given cell type according to growth phase, cell cycle position and differentiation status. Table 1 below shows typical values for the in vitro dimensions of adherent mammalian cells, measured as suspension forms (typical range being 15-25 μm diameter and >200 μm² cross-sectional area), with the smaller dimensions of non-adherent human blood cells included as reference values.

TABLE 1
the size parameters for various mammalian cells

| | | | Volume | | |
|---|---|---|---|---|---|
| | | | mean cell | Nominal | |
| Cell line | | | diameter | cross-sectional | |
| designation | Origin | Type | (μm) | area (μm²) | ref. |
| Caco-2 | human | Colorectal carcinoma | 16.9 | 224 | a |
| HT-29 | human | Colorectal carcinoma | 17.3 | 235 | a |
| HT29D4 | human | Colorectal carcinoma | 15 | 177 | a |
| CCD 841 CoTr | human | SV40 transformed colon cell | 19.2 | 290 | a |
| | | line | | | |
| HuTu 80 | human | small intestinal carcinoma | 16.7 | 219 | a |
| NCI-H292 | human | lung carcinoma | 16.2 | 206 | a |
| NCI-H460 | human | lung carcinoma | 18.2 | 260 | a |
| NCI-H661 | human | lung carcinoma | 25.3 | 503 | a |
| BEAS-2B | human | SV40 transformed lung cell | 15.9 | 199 | a |
| | | line | | | |
| MCF7 | human | breast carcinoma | 17.3 | 235 | b |
| HeLa | human | cervical carcinoma | 22 | 380 | c |
| mESC | mouse | embryonic stem cells | 20 | 314 | d |
| lymphocytes | human | Human blood | 7.2 | 41 | b |
| monocytes | human | Human blood | 9 | 64 | b |

Refs:
a: Franssen-van Hal et al. (2005) Archives of Biochemistry and Biophysics 439 32-41
b: Chalmers et al. (1998) Biotechnology and Bioengineering 59 10-20
c: Shi et al. (2004) Biotechnology and Bioengineering 41 745-754
d: Rosenthal et al. (2007) Biomaterials 28 3208-3216

Micro-featured surfaces (e.g., cell carriers or ‘microwells’) have been used to limit the extent of cell movement in adherent and non-adherent cultures. This approach reduces or precludes cell immigration and emigration from a microwell to allow the analysis of single cells. Deutsch et al. have described cell carriers and methods of selection that address the need for the analysis of single cells at specific locations using methods suitable for non-adherent cultures (U.S. Pat. No. 5,506,141, U.S. Pat. No. 5,310,674, U.S. Pat. No. 5,272,081 & U.S. Pat. No. 4,729,949). The cell carrier concepts of Deutsch et al. have led to products developed by Molecular Cytomics (www.molecular-cytomics.com) based on technology developed at Bar Ilan University, Israel (Deutsch et al. Biomed Microdevices. (2006) 8 361-374; Deutsch et al., Lab Chip, 2006, DOI: 10.1039/b603961h). Again, in such micro-wells cell orientation is neither ordered to enhance the means of analysis nor constrained to allow the analysis of cells with a deduced divisional history. Such microwells could be used for adherent cells, depending upon the biocompatibility of the substrate but do not intrinsically control cell contacts. Further, the capacity of the well is modified by changing at least two dimensions thereby modifying the local impact of dimensional constraints to proliferation.

A further approach has been to constrain the growth of cells to a given location using modified surface properties such as the micropatterning of the surface with adhesion promoting or rejecting properties (usually in island based formats). A resultant problem is that the cell now undergoes changes associated with the adhesion responses and such selective capture does not reveal divisional history. Huberman et al. (U.S. Pat. No. 6,495,340) has described improved cell carrier grids—capable of containing and retaining individual living cells at discrete locations substantially in a single focal plane so that accuracy of data collected is increased. According to some embodiments of the Huberman invention, the body is at least partially coated with a biologically active material. However, within such micro-well formats, cell orientation is not ordered to enhance the means of analysis (temporal order of events) or to allow the reporting of a cell with a deduced divisional history.

Micropatterning of a substrate by the application of adhesion promoting and rejecting aspects to the surface has been used to modify cell attachment and adhesion to control cell location. For example Yamato et al. have described the rendering of regions of a substrate as non-adhesive (i.e., “non-fouling”) by suitable chemical modification (Yamato et al. (2003) J Biomed Mater Res A. 67 1065-71) allowing the patterned substrate to be used for the preparation of cell arrays. De Silva et al. have described the use of polydimethysiloxane (PDMS) stamps to create adhesion promoting patterns of chemicals (e.g., laminins or collagen) on PDMS substrates exploiting the natural hydrophobic tendency of PDMS to reduce adhesion potential (De Silva et al. Biomed Microdevices (2004) δ 219-22). PDMS consists of repeating units of —Si(CH₃)₂—. PDMS is a durable elastomer, deformable, homogeneous and isotropic. In addition, it is inexpensive, flexible, optically transparent to wavelengths greater than 230 nm, impermeable to water but not to gases and is non-toxic to cells. It is recognised that the surface properties of PDMS can be modified (e.g., by plasma oxidation resulting in the introduction of silanol groups (Si—OH) on the surface by oxidation of methyl groups (Si—CH₃) of PDMS at the plasma/polymer interface) with a reduction in hydrophobicity, modulating the potentially cell adhesion rejecting properties of PDMS.

Micropatterning has been widely investigated in respect to biocompatibility of surfaces. Cell responses to typographically patterned surfaces (e.g., on biodegradable polymers or implant surfaces) have been explored for the purpose of developing biomechanical structures and wound healing materials (U.S. Pat. No. 5,833,641 & U.S. Pat. No. 4,832,759). Microtopography can contribute to changes in cell behaviour, including orientation, locomotion, speed, direction and polarisation (for review: Curtis & Wilkinson (1997) Biomaterials 18 1573-83). Such changes are deemed important for the performance of materials in terms of biocompatibility of implants and in tissue engineering applications. Curtis & Wilkinson (1997) noted the impact of single cliffs, converging/diverging cliffs, groove/ridges, dots, spikes, hills, pits, tunnels and tubes, cylinders, mesh and random roughness. With a focus on implants and therapeutics, Curtis & Wilkinson (1997) and, more recently, Freitas (Freitas (2003) Nanomedicine, Volume IIA: Biocompatibility, Landes Bioscience, Georgetown, Tex.), have noted that a wide range of cell types are capable of responding to typographical cues include: chondrocytes, endothelia, epithelia, fibroblasts, leucocytes, lymphocytes, macrophages, mesenchyme, neurons, osteocytes, oligodendrocytes, smooth muscle cells and some tumour cells. Microtopographical cues for constraining the degrees of freedom for a cell based assay as noted above will be different from cues that provide optimal cell orientation and growth for implant biocompatibility and tissue engineering applications.

A further example of micropatterning with respect to biocompatibility (WO2006/114098) primarily relates to the use of micropattern topographical features on a surface to be used as a surgical bone implant, the aim being to promote bone growth. However, the inventors also disclose that the use of a surface having micrometer scale topographical structures promotes the growth of, for example, undifferentiated embryonic stem cells. The lateral dimension of each topographical structure is said to be 0.1-10 μm. In the case where the topographical structures are parallel rows of short, straight ridges, the spacing between the ridges is 2, 4 or 10 μm and the structures are only shown to promote mineralisation of osteogenic MC3T3 cells. The disclosed dimensions are smaller than a typical cell diameter (see table above; mean diameter 18.2 μm, std dev=3.0 μM; n=11) and cells growing on these surfaces would not be able to locate themselves exclusively between the raised topographical structures but would, rather, spread across the micropatterned surface using the environment to promote a differentiation (mineralisation) rather than ordered proliferative behaviour.

### Limitations of Existing Cell-Based Assays for the Evaluation of Cell Proliferation.

Cell proliferation can be assessed by simple means such as the counting of cells in cultures at discrete time points or the determination of a related parameter such as the frequency of cells demonstrating cell division (e.g., mitotic index). A ‘clonogenic assay’ is a typical example of a cell-based assay that can be performed using adherent cells attached to a substrate or non-adherent cells suspended in a semi-solid support medium. ‘Clonogenic potential’ is a biological concept of widespread interest in assay design, namely that a given cell may have the potential to proliferate and give rise to a ‘colony’ of cells in which some daughter cells from each generation retain the potential to proliferate. This is a concept readily understood in microbiological assays for the isolation of monoclonal colonies. Clonogenic potential is a fundamental descriptor of viability and is commonly used in reference to cellular characteristics both in vitro and in vivo. For example, clonogenic potential is of interest in the investigation of stem cell properties regarding heterogeneity, lineage plasticity, clonal fluctuation and cell-environment interactions.

Clonogenic potential may be limited according to cell type. For example, human diploid cells derived from normal tissue undergo a characteristic pattern of primary cell growth in cell culture, ending with a non-replicative phase (“senescence”) and, finally, cell death. Transformation (e.g., by the DNA tumour virus simian virus 40 [SV40]) increases the growth potential of human diploid fibroblasts and cells transformed near the end of their normal in vitro life span are able to proliferate transiently. Neoplastic cells (e.g., derived from a malignant cancer) characteristically show proliferation activity beyond the natural limits demonstrated by primary cell cultures. According to the type of culture, cells can become limited in their proliferation potential by local conditions such as growth factor exhaustion and the operation of contact inhibition. In vitro culturing of cells to test their clonogenic potential is in common practice (e.g., neoplastic cells can be used in clonogenic assays to test their radiosensitivity, chemosensitivity and the probable clinical efficacy of a therapeutic agent).

Attempts to identify a specific cell and to follow that cell's future divisional or other behavioural patterns (e.g., clonogenic potential) have previously been approached by, for example, dilution of adherent cells to allow attachment at separated locations for progenitor cells to aid clonal identification by the formation of colonies at those locations. This approach has the draw-back that a clonogenic assay typically requires 1-3 weeks of incubation to stabilise a macroscopic colony and uses low plating densities that can both compromise clonogenicity and restrict the ability to identify progenitors in sparse fields. Problems also relate to the exhaustion of medium during cultivation and the production of satellite colonies due to cell detachment and re-attachment at a different location. Some highly motile cells can act to diffuse colony structure and make colony recognition problematic.

To address issues of the relationships and interactions between cells and dynamic features such as motility and cell division, time-lapse imaging provides a ‘gold’ standard in which individual cells are tracked at set intervals and their divisional history, inter-relationships and behaviour reported. The resolving power for monitoring a given event is determined by image sampling intervals. The approach is labour-intensive for manual and semi-manual tracking and can potentially result in deleterious effects on cell cultures due to the high frequency of light exposures required to capture and therefore register a transient event such as cell division. The sampling interval is determined by the ability to track individual cells and parameterise according to the cellular behaviour (degrees of freedom). For example a fast moving cell must be sampled more frequently than a slow moving cell in order to identify and track behaviour. The optimal sampling interval for any one degree of freedom is determined by Nyquist sampling. Time-lapse imaging typically generates large amounts of image-based information which results in problems for short- and long-term data storage, analysis and access. Furthermore, the acquisition platform is often continuously occupied, resulting in limitations for assay throughput. Reducing the degrees of freedom (e.g., constraining motility) or introducing aspects of order and patterning in such an assay would reduce the impact of confounding factors (e.g., the need for continuous observation to track events associated with a given cell in order to prevent confusion over cell identity).

To enhance scale, throughput and assay efficiency, there have been attempts to array cells at defined locations. Micro-well plates are in common use for cell-based assay, providing coherent locations for optical analysis. The ‘cell carriers’ of Deutsch et al. can be arrayed in regular patterns providing addresses for analytical events. Various types of cell array formats have been described previously for non-adherent cells. For example, Deutsch et al. (Lab on a Chip 2006, DOI: 10.1039/b603961h) described glass slides having wells of diameter 15, 20, 100 and 200 μm and depth 6, 8, 14 and 35 μm, respectively, achieving 16,000, 9000, 360 and 55 wells per slide, respectively. Love et al. (Nature Biotechnology (2006) 24 703-707) described PDMS slides having wells of diameter 50-100 μm and depth 50-100 μm, achieving 30,000-40,000 wells per slide. Yamamura et al. (Analytical Chemistry (2005) 77 8050-6) described a polystyrene slide with 200,000 wells per slide, of 10 μm diameter and 12 μm depth. Biran & Walt (Analytical Chemistry (2002) 74 3046-54) described glass fibre optic bundles with fibres of 2.5 or 5 μm and 50,000 or 24,000, respectively, fibres per bundle.

The dimensions of the microwells described in these publications cover the range of dimensions from below than to greater than that of a somatic human cell (nominally 15 μm in diameter when measured in a near-spherical conformation but with an extensive range of diameters according to type). In all cases a micro-well dimension is fixed for an assay purpose, as distinct from the user using a range of dimensions within a micro-featured array to select a constraining influence for a given analytical purpose or to provide for multiple assay types within an arrayed surface.

A number of documents describe the use of grooves to assist with the growth of cells (see for example, WO 95/22305, US 2005/0177231, WO 01/81552 and WO 2004/034016). Furthermore, there are a number of documents that describe the use of microtextured and microgrooved surfaces for studying cell migration, cell growth and cell morphology (see for example, Bhavani et al., Cell Motility and the Cytoskeleton, 64, 1-5, 2007; den Braber et al., Biomaterials, 17, 2037-2044, 1996; Matsuzaka et al., Biomaterials, 24, 2711-2719, 2003; Hsu et al., Biotech. Bioeng., 92, 579-588, 2005; and Dalton et al., J. Biomed. Mater. Res., 56, 195-207, 2001).

There exists a continuing need to improve cell culturing and observation techniques so as to enhance the ability to track individual cells and groups of cells with certainty and to determine more easily their downstream fate. Despite increased data storage capacity available within computing systems, there is a continuing need to reduce the expanding volume of data collected in conventional assays. A further continuing need is to provide simple methods of constraining the potentially confounding degrees of freedom presented by systems with microfeatures that impact in a complex manner upon cellular behaviour.

### SUMMARY OF INVENTION

According to a first aspect of the invention there is provided a method of determining the proliferative potential of a cell comprising the steps of allowing the cell to adhere to a microtrench that restricts movement of the cell such that on cell proliferation one or more daughter cells are sequentially arranged in a line, observing the microtrench on at least two occasions and determining whether a cell has undergone proliferation.

The method according to the first aspect of the present invention may further comprise the step of exposing the cell to a test agent. The test agent may be one having a known or anticipated effect, such as to cause cell death, or may be an agent of interest whose effect is unknown, e.g., a newly developed drug candidate compound. The test agent may be, therefore, by way of non-limiting example, a small chemical entity, a biological molecule, an artificially modified biological molecule, a naturally occurring substance, an artificially synthesised substance, an antibody, an organic or inorganic chemical compound or any form of radiation (e.g., UV radiation). Molecules and compounds may be modified by the addition of, for example, fluorogenic, bioluminescent, chemiluminescent or radioactive entities.

The term “proliferative potential” refers to the ability of the cell to proliferate, the rate of proliferation, and also clonogenic potential. In a preferred embodiment of the present invention the method is used to determine whether a cell exhibits minimum clonogenic potential (MCP). MCP is defined as the ability of a cell to divide and that at least one product of the cell division is capable of completing a further mitotic division, i.e., the cell is capable of giving rise to a daughter cell which is capable of also giving rise to a daughter cell.

A cell exposed to a test agent may be observed after a given time, e.g., after one hour, five hours, 10 hours, 24 hours, 1 week. The time interval selected would preferably be in excess of a cell cycle period (nominally >22 h for U-2 OS cells) to observe cell cycle perturbations, greater than two cell cycles (nominally >44 h for U-2 OS cells) to observe minimal clonogenic potential, over shorter time frames to assess the induction of acute cell death, or over very short intervals (e.g., <5 h) to assess cell movement. The skilled person would readily be able to determined appropriate time intervals according to the cell type used.

The cross-sectional area or width of the microtrench restricts cell/cell passing during interphase, thereby ordering the directional axis of division such that daughter cells segregate in alignment with the microtrench longitudinal axis. Thus, following division of a progenitor, the resultant daughters are next to each other. Logic then allows for a microtrench initially occupied by a single cell to report, for example, a division (two cells found at a previous location occupied by only one cell) or subsequent division of each daughter (four cells found at a previous location occupied by only one cell).

It is preferred that the method comprises ensuring that only a single cell, or only a few cells (e.g., 2 to 5 cells) are adhered to the microtrench. If a large number of cells are adhered to the microtrench, then it becomes very difficult to determine whether a specific cell has undergone cell proliferation. The microtrench may be defined on or in a surface of a substrate. Preferably, the ratio of the width of the microtrench to the maximum length of the short axis of the cell is about 6 or preferably less, wherein the maximum length of the short axis of the cell type is measured when a cell is in attached or adherent or rounded mitotic states. Alternatively, the ratio of the cross-sectional area of the microtrench to the maximum cross-sectional area of the cell is about 2 or preferably less, wherein the maximum cross-sectional area of the cell type is measured when a cell is in detached or in a suspended form prior to attachment. In use, the dimensions of the trench allow for gravity sedimentation of cells (from an appropriately prepared suspension) onto the micro-trenched substrate, the attachment of one or more cells to a surface of the trench and prevent each of the one or more cells or their mitotic progeny from passing another of the one or more cells within the trench. Alternative methods of delivering cells to the microtrenched area, e.g., microfluidic, centrifugation, electrostatic adherence and printing methods, may be used. The restriction in passing maintains the order of cells in the microtrench for analytical advantage. The surface adhesion properties of the substrate may be modified to permit enhanced cell motility enabling cells to find preferential attachment and spreading and alignment within the microtrench.

In some embodiments of the method according to the first aspect of the present invention, more than one cell may be present within the microtrench. Furthermore, the one or more cells located within a trench may be of one or more different cell types.

The cell culture microtrench presents a means of imposing dimensional, topological and tuneable substrate constraints on cellular behaviour for analytical advantage. Analysis of cell behaviour, especially proliferative potential, can be undertaken by various microscopical methods. Advantageously, the use of a microtrench overcomes many of the problems associated with growing cells in culture for the purposes of observing their behaviour, in particular their life cycle and studies of clonogenicity. As a comparison with some of the various difficulties set out above in relation to known techniques, the use of a microtrench overcomes these as follows:

Cell proliferation: The microtrench acts to constrain the randomisation of cell location following cell division as the result of the fact that cells proliferate along the longitudinal axis of the trench, i.e., there is not sufficient width in the trench for cells to grow side by side across the width of the trench.

Cell motility: The microtrench acts to constrain motility along a preferred axis, since there is not sufficient width in the trench for cells to pass one another, preserving cell order.

Cell loss: The microtrench acts to identify cell loss by allowing for the reporting of the frequency of occupancy at a fixed location and the subsequent attrition, as a result of being able to determine the constrained location such as a given microtrench at which a cell was previously found.

Cell incursion: The microtrench acts to allow for the evaluation of cell entry into an observed field by allowing for the identification of the adventitious occupation of previously empty trenches. The microtrench acts to constrain incursion so that invasion into an assay area can be prevented or reduced.

Cell morphology: The microtrench acts to constrain morphological changes by reducing local motility as the result of the physical constraints provided by the microtrench. However, the invention allows for the major changes in shape (e.g., during cell death or mitosis) to occur. The invention provides structural patterning whereby the cell morphology is altered by the trench structure, providing further routes to image analysis algorithms.

Cell-to-cell heterogeneity: As mentioned above, in cell-based assays cell-to-cell heterogeneity can be a confounding factor or a feature of interest such that analysis of single cells must be carried out. The invention acts to reduce this confounding effect by enabling single cell identification and analysis as a result of being more readily able to locate a specific cell for a second or subsequent observation.

Microniche exhaustion of growth and survival factors: The microtrench allows cells to be identifiable, separated and with maximal access to common medium conditions, whilst at the same time enabling cells to experience optimum cell-to-cell contact within a microtrench and to have sufficient access to analytical reagents.

Low event frequency: The microtrench allows for high relative packing densities by varying the periodicity of microtrenches.

Cell-cell interaction: The microtrench allows cellular interactions to be manipulated within one dimension (e.g., end-to-end contact within the microtrench) or manipulated (e.g., co-cultivation of a cell type A, optimally constrained within a microtrench, with a cell type B that shows random motility over that specific microtrenched surface—thereby providing discrete and addressable points of contact of the two cell types). Varying the relative cell densities can allow different forms of assay. However, depending on the trench characteristics, cross-trench cell-cell contact can be established and maintained.

Cell lineages and inter-relationships: The microtrench allows a cell lineage to potentially form within a microtrench with preservation of divisional order and relationships within 1 dimension.

Cellular substrate-attachment and substrate-adherence potential: The microtrench may comprise a substrate surface that imparts a degree of plasticity to cell form and behaviour such that microtopological and surface chemistry features can impart constraining influences on cells for the purposes of an assay.

The method of the present invention enables a user to select microtrench dimensions that can apply a constraining influence on the degrees of freedom pertaining to a given substrate characteristic (e.g., use of a substrate that reduces the extent of full adhesion of a given cell type to the surface as selected by the user) and the degrees of freedom arising from cell-associated characteristics (determined by the type of cell selected by the user). In practice the user would typically search for the appropriate level of constraint (e.g., aligned attachment and non-passing proliferation) within a microtrench array that offers different combinations of trench dimensions. A person skilled in the use of a microscope and cell observation would readily be able to carry out such a search. Having selected the appropriate microtrench conditions, the analysis can be restricted to those substrate addresses with microtrenches having the selected characteristics or alternatively the assay can be transferred to a microtrench array in which those characteristics are reproduced by multiple trenches.

The microtrench used in the present invention is preferably for use with one or more cells of the same cell type. The ratio of the cross-sectional area of the trench to the maximum cross-sectional area of the cell type may be about 2, 1.9, 1.8, 1.7, 1.6, 1.5, 1.4, 1.3, 1.2, 1.1, 1, 0.9, 0.8, 0.7, 0.6, 0.5 or, preferably, less than 0.5, the maximum cross-sectional area of the cell type being measured when a cell is in detached or suspended form. Typical values for the range of diameters of a human adherent cancer cell (e.g., U-2 OS osteosarcoma cell line), measured following detachment and adopting a nominally spherical morphology, is 15-30 μm.

The ratio of the width of the trench to the length of the short axis of the cell type, measured when a cell is adherent and aligned within a microtrench with its longer axis substantially parallel with the long axis of the microtrench, is preferably less than 6.0 and more preferably less than about 5.5, 5.0, 4.5, 4.0, 3.5, 3.0, 2.5 or 2.0 and may be about 1.9, 1.8, 1.7, 1.6, 1.5, 1.4, 1.3, 1.2, 1.1, 1.0 or, preferably, less than 1.0. A typical value for the length of the axes of a human adherent cell (e.g., U-2 OS osteosarcoma cell line), measured in an adherent interphase state is 10-30 μm for the short axis and >30 μm for the long axis. A typical value for the diameter of a human adherent cell's nucleus (e.g., U-2 OS osteosarcoma cell line), measured in an adherent interphase state is 5-10 μm and frequently adopting a flattened ovoid shape.

The microtrench for use in the present invention is preferably for use with one or more cells of the same cell type. The ratio of the length of the trench to the maximum length of the long axis of the cell type is at least 1 and preferably greater than 2 to accommodate multiple cell occupancy. A typical value for the length of the long axis of a human adherent cancer cell (e.g., U-2 OS osteosarcoma cell line), measured in an adherent interphase state is >30 μm for the long axis.

Preferably the microtrench is less than 550 μm in length, more preferably between about 100 and 512 μm in length, and most preferably about 500 μm in length. This length allows a single trench to be conveniently imaged at appropriate resolution using transmission in a single field of view, this field being typically about 512×512 pixels (e.g., representing a field of 512 μm×512 μm), for example. However, the microtrench may be replicated so that 2 or more microtrenches fill the field of view. Furthermore, the preferred length provides sufficient space for the accrual of events during the typical period of a short-term proliferation assay (approximately 72 h). Microtrenches that are longer than 550 μm also have the disadvantage that interference from other cells contained within the trench may confuse any analysis. By restricting the length of the microtrench, such a risk is reduced. Each end of the trench is formed such that cell entry by motility-based immigration is prevented. For example, one or more ends of the trench may be defined by an end surface or wall or by the tapering of the microtrench to prevent the passage of a cell from an adjoining microtrench.

In some embodiments, the trench may have a width of about 5-35 μm, preferably 8-30 μm, more preferably 10-25 μm, yet more preferably 12-20 μm and most preferably about 14 μm or about 16 μm.

A cell is in detached or suspended form when it has few contacts, preferably no contacts, with a surface, such that the cell is able to move freely throughout the supporting medium and is therefore likely to adopt a nominally spherical form. A cell is in an attached or adherent form when there is a degree of bonding to a substrate (for example, at least one point of contact) and the cell is no longer free to move throughout the supporting medium but may undertake movement over the substrate through a process of cell motility. The attached or adherent state is readily determined by the skilled person using, for example, microscopic observation. In the attached or adherent states a cell may change its morphology to more flattened forms by spreading the plasma membrane over the substrate such that its dimensions may be conveniently described as a cell diameter measured along either the shortest or longest axis of the cell form. Preferably the short axis extends in a direction across the width of the microtrench and the longer axis extends in a direction along the length of the microtrench. The microtrench may be formed as a depression in the surface of the substrate, each side of the trench being formed by a wall. Alternatively, the microtrench may be defined by two or more walls projecting from the surface. In a further alternative, these two options may be combined, such that the microtrench not only forms a depression in the surface but is also further defined by means of two or more walls which project from the surface. In any case, it is preferred that each wall has a wall top surface formed so as to discourage and, most preferably, prevent, cell adhesion to the wall top surface.

The wall top surface may be 1-30 μm wide, preferably 5-25 μm wide, more preferably 10-20 μm wide. In a more preferred embodiment, the wall top surface is about 12 μm or 15 μm wide.

Alternatively or additionally, the material forming the wall top surface may comprise and/or be coated with a compound which discourages cell adhesion to the surface.

The term “wall top surface” is intended to indicate a surface of a wall which does not form part of an interior surface of a microtrench. In an example embodiment, a trench has a substantially rectangular cross section, with each substantially vertical trench side being defined by a wall. The wall between any two microtrenches will have two substantially vertical sides, each forming the side surface of adjacent trenches, with the sides being separated by a substantially horizontal surface; it is this surface which is the “wall top surface” and is, therefore, the surface which spaces one microtrench from an adjacent microtrench.

The cross-sectional shape of the microtrench may be, by way of non-limiting example, generally square, rectangular, V-shaped, U-shaped, semi-circular, ovoid, having canted sides or being stepped.

The microtrench may have a depth which prevents motility-based emigration of each one or more cells from the trench. The depth may be sufficient to prevent a cell which is attached but in motile interphase from passing another cell which is attached but in motile interphase. Preferably, the depth of the trench may be about 1, 2, 3, 4 or 5 μm, more preferably about 1.8 μm.

The term “motile interphase” indicates that a cell is in the interphase part of the cell cycle, a state which is readily determined by the skilled person using, for example, microscopic observation. When in this state, an adherent cell maintains dynamic contacts with a surface but is mobile or motile, i.e., able to migrate over the surface by an active process.

The microtrench may have a width of 5-35 μm, preferably 8-30 μm, more preferably 10-25 μm, yet more preferably 12-20 μm and most preferably about 14 μm or 16 μm. The microtrench may be defined by two or more walls each having a wall top surface which is 1-30 μm wide, preferably 5-25 μm wide, more preferably 10-20 μm wide, most preferably 12 μm or 15 μm wide.

The dimensions of the microtrench are such that for cells measured in pre-attachment suspension form, the ratio of cross-sectional area of the trench (from 50 μm wide and 5 μm deep to 10 μm wide and 1 μm deep) to the cross-sectional area of a typical cell would range from 1.25-0.05 (for a cell of 200 μm²), 0.83-0.033 (for a cell of 300 μm²) and 0.625-0.025 (for a cell of 400 μm²). Preferably, for cells measured in pre-attachment suspension form, the ratio of cross-sectional area of the trench (from 30 μm wide and 2 μm deep to 20 μm wide and 1 μm deep) to the cross-sectional area of a typical cell would range from 0.3-0.1 (for a cell of 200 μm²), 0.2-0.067 (for a cell of 300 μm²) and 0.15-0.05 (for a cell of 400 μm²).

The dimensions of the microtrench are such that for cells measured in pre-attachment suspension form, the ratio of the width of the trench (for 5-30 μm wide) to the length of the short axis of a typical cell, comprising a relatively fluid cytoplasm and a less deformable nucleus, would range from 1-6 (for a cell of 5 μm diameter), 0.5-3 (for a cell of 10 μm diameter), 0.33-2 (for a cell of 15 μm diameter), 0.25-1.5 (for a cell of 20 μm diameter), 0.2-1.2 (for a cell of 25 μm diameter) and 0.167-1 (for a cell of 30 μm diameter).

Preferably, the microtrench has an interior surface of the trench comprising and/or being coated with a compound which encourages cell adhesion. Alternatively or additionally, an interior surface of the trench may comprise and/or be coated with a compound which alters the behaviour of one or more of each one or more cells. By way of example (but not of limitation), a compound might promote cell death, cause cell cycle arrest, change cell proliferation, or result in alterations in cell morphology and motility. The compound may be one which has known properties (i.e., known effects on the behavior of cells), or one which is being screened to determine its properties (e.g., whether it causes one or more changes in cell behaviour or morphology).

In a preferred embodiment, the wall top surface is such that cell attachment and motility within the microtrench is preferred and/or, once established, is constrained. In a preferred embodiment, the microtrench dimensions offer a preferential adhesion environment that imposes alignment on the cell and constrains motility and emigration from the microtrench.

The constraining effect of the microtrench on cell passing arises, in part, from: the restricting nature of the physical dimensions of the microtrench relative to cellular dimensions, the restricting effect of occupancy by one cell on the ability of a second cell to form attachments for cell passing and the restricted compressibility of a cell's nucleus when compared to the more fluid cytoplasm. It is a common observation that an interphase cell's nucleus has restricted compressibility or reduced ability to deform when compared to the more fluid properties of the surrounding cytoplasm. This provides for a non-linear relationship between the overall dimension of a cell (e.g., cross-sectional area or length of short axis) and the contribution of that parameter to a restriction of cell passing within a microtrench. Effectively, the cell's nucleus provides a restrictive minimal width of a cell located in a microtrench. Such a minimal width can, therefore, be determined by the nominal diameter of a cell's nucleus, e.g., 5-10 μm for a U-2 OS osteosarcoma cell, measured in an adherent interphase state.

The microtrench presents general topological features that promote attachment and spreading and alignment acting to constrain the cell-associated characteristics that contribute degrees of freedom to a given assay. Additional substrate-associated features can contribute attachment/adherence degrees of freedom to a cell adherence process and thereby impact upon the importance or otherwise of the cell-associated degrees of freedom. For example, a cell with highly adherent properties may be capable of successful attachment and spreading on a relatively hydrophobic substrate, while cells with lower intrinsic adherent properties could be excluded from such surfaces. The microtrench provides a substrate surface that can be modified to have variable characteristics for the adherence or attachment of cells, imparting different degrees of plasticity to cellular behaviour, such that topological features (e.g., microfeatures such as microtrench pitch) can then be used to direct the forms of cell attachment, adherence and alignment.

The cell-associated features range from general surface charge properties to the expression of specific adhesion molecules and are determined by the assay and user requirements. The substrate surface properties range from chemical modification of hydrophobicity to the micropatterning of biological molecules involved in cell adhesion signalling and allow for a tuning of the degree of plasticity sought. For example, the surface adhesion properties of the substrate may be modified or remain unmodified to permit enhanced cell motility or attachment-detachment dynamics enabling a cell to find preferential attachment, spreading and alignment, according to the presentation of topological cues in the domain of cellular size. Patterned cues provide a specified approach to constrain aspects of the degrees of freedom for cell-associated features for a given assay purpose in addition to providing an addressable location on the substrate.

The microtrench may be provided as an array comprising two or more substantially parallel microtrenches, preferably a plurality of substantially parallel microtrenches, wherein each microtrench is separated by a wall having a wall top surface.

Each wall top surface is preferably arranged so as to prevent the movement of a cell from one wall top surface to another wall top surface. In a preferred embodiment, the width of a wall top surface is such that cell attachment and motility within the microtrench is preferred or, once established, constrained. The microtrench dimensions offer a preferential adhesion environment that imposes alignment on the cell and constrains motility and emigration from the microtrench. Such a preferential environment acts to reduce emigration from the microtrench and subsequent cell incursion into adjoining microtrenches.

For example, each wall top surface may be 1-30 μm wide, preferably 5-25 μm wide, more preferably 10-20 μm wide and most preferably about 12 μm or 15 μm wide. Alternatively or additionally, the material forming each top wall surface may comprise and/or be coated with a compound which discourages cell adhesion to the surface.

The array may further comprise one or more markings which allows the user to determine the orientation of the array and/or the location within the array of each at least one microtrench. This may be achieved by, for example, symbols placed on the wall top surface of one or more walls which separate any two microtrenches. Such symbols could appear on the wall top surface of every wall which is included within the array. The symbols may be visible by any routine means, for example by use of microscopy with or without the additional use of fluorescent or other imaging methods. The markings may also be utilised by an automated orientation/location system, e.g., by use of a barcode or other automatically detectable pattern.

This enables the user of the array, when observing the contents of a microtrench, to use the markings to determine which microtrench within an array is being observed, thereby improving the ability to find a previously observed cell or cells within a particular trench and enabling the user to more readily determine whether the cell or cells have, for example, undergone any division, migration and/or apoptosis events.

The microtrenches within the array have dimensions that constrain aspects of cell behaviour:
A cross-sectional area that will accept the attachment of rounded suspension preparations of cells and permit a cell to initiate attachment to the substrate within the microtrench (i.e., favourable microtrench cell adherence properties). As mentioned above, preferred dimensions of the microtrenches are guided by the general principle that the ratio for cross-sectional area of the microtrench to the maximal cross-sectional area of a typical live cell, measured in detached/suspension form, is less than 2, or that the ratio for the width of the microtrench to the length of the short axis of the live cells, measured in attached or adherent or rounded mitotic states, is about 6 or preferably less.
A width of wall at the top surface that does not favour cell attachment to that surface in preference to the micro-trench (i.e., low wall upper surface adherence).
A periodicity of microtrenches (as defined by the wall width) that does not favour wall-to-wall migration of cells (i.e., low bridging of microtrenches).
A depth that is sufficient to constrain the location of adherent cells to within the confines of the microtrench with minimal escape from the microtrench (i.e., low motility-based emigration).
A depth that is sufficient to constrain the movement of attached but motile interphase cells to movement patterns that align with the axis of the microtrench but maintain the order of cells within a microtrench (i.e., parallelised cells with low bypass or low contra-flow within the microtrench).
A restricted cross-sectional area at the end of the microtrench and a width at the open ‘top’ surface of the microtrench that tend to disallow cell entry by motility-based immigration.

The microtrenches within an array may have varying dimensions to one another. For example, an array may comprise microtrenches which have widths which differ from one another. The use of such an array would allow the user to determine which microtrench dimensions result in the desired location and in-trench localisation of cells.

The invention represents a novel approach to the determination of information relating to the proliferation potential of a cell and other divisional or behavioural characteristics, by imposing a means of constraint and preserving cell order (by microtrenching) on adherent cell behaviour. The invention complements and overcomes some of the shortcomings of traditional time-lapse imaging. A surface is patterned in the form of microtrenches either within a substrate or as raised microtrenches continuous with the substrate, isolated from each other by raised walls. The patterns of microtrenches can vary but a preferred form is to have arrays of microtrenched areas with parallel and fixed numbers of microtrenches to aid microtrench address recognition.

According to a preferred embodiment, the method according to the first aspect of the present invention comprises attaching adherent cells (e.g., mammalian cells) within one or more microtrenches to provide low occupancy rates, preferably one initial cell per microtrench, providing a ‘reporting’ microtrench occupied by a progenitor cell. Prior to cell attachment, source cultures may be asynchronous with respect to cell cycle age or synchronised to increase the frequency of, for example, division events occurring within an assay period. Following cell attachment, cell division, loss or proliferation arrest of the progenitor or daughter cells can reveal patterns of interest for assay purposes. The surface properties of the material forming the trench permits the adhesion, proliferation and motility of eukaryotic cells normally capable of such behaviour during adherent in vitro growth. The arrangement of microtrenches within an array according to a preferred aspect of the invention provides for a control of cell behaviour by ordering cell attachment, motility and division, such that cells ‘track’ along the axis of a given microtrench, providing an orientation and ordering of cells on an array.

Automated analysis of light-microscope images of cells in prior art methods is often hampered by the variability in cell appearance and in their positional relationship. Image analysis techniques such as thresholding or linear and radial edge detection filters are usually followed by more sophisticated algorithms such as the watershed algorithm or a Hough transform (e.g., CHARM) in order to segment cells from their background. Over- or under-segmentation regularly occurs and region splitting and merging techniques are usually employed to counteract this. The invention circumvents many of these problems, by introducing a predictable structure to the environment such that, upon adherence, cells preferentially extend along the trench axis. The introduction of a predictable structure permits a comparison to be made of, for example, images of that structure with and without cells for the purpose of determining the interference imposed by the presence of cells. Such interference could be used to detect microtrench occupancy or changes in occupancy with time by comparing a sequence of images of the same field gained at different times. Such determinations could be carried out by a person skilled in the art of Fourier analysis (e.g., in which a Fourier transform would be used to define the relationship between a signal in the time domain and its representation in the frequency domain).

Tracking a cell's motility is also made easier through the use of the invention as its motion is also constrained once located within a microtrench. Tracking can only be achieved if cell correspondence can be determined between time points. Using prior art systems, this process is hampered by increasing degrees of freedom; if a cell is allowed to move in two dimensions as well as change its morphology, appearance and brightness significantly, correspondence between a first and subsequent observation is difficult. The microtrench of the invention reduces the degrees of freedom by constraining morphology but, most significantly, restricts motion to a single dimension, such that the problems of cells passing, overlapping and/or changing direction are all suppressed.

The low profile (i.e., small depth) nature of each microtrench acts to organise the behaviour of cells by the orientation of adhesion at normal and favourable cell densities for in vitro culture, without imposing microniche environments that could restrict cellular access to growth factors or exogenously supplied reagents associated with a specific analysis.

The ability to minimise the passing or contra-flow of cells within a microtrench maintains an order for the products of cell division which can inform cell division history. The microtrench maintains the order of multiple cells tracking along a trench at a readily identifiable location, allowing a cell or its progeny to be revisited and identified as part of a process or analysis, without the need for continuous observation of cell movement or division events.

The microtrenching constraints upon cell behaviour are such that, upon culture, the frequency and order of cells within the trench can be used to identify cells with clonogenic potential or expressing sustained cell cycle arrest.

The pattern of microtrenches within an array aids cellular analyses through algorithm-based identification of cellular events within the micro-trenches, within images or signals, acquired optically or otherwise. The properties of the array allow optical imaging techniques to be used in trans-illumination (through the sample) and under epi-illumination (e.g., fluorescence). The microtrench pattern provides locations for the programmable analysis of cells at specified addressable locations, thereby reducing analysis time. Large area array imaging may be used, by the application of a CCD (charge coupled device) or a CMOS (complementary metal oxide semiconductor) image sensor, to image a whole microtrench pattern or a region of that pattern. Addressable image sensors would provide for discrete imaging of an aspect of microtrench pattern at given locations to enhance assay flexibility or efficiency. Addressable locations also provide for cross-modality imaging of the same sample such as light microscopy and subsequent scanning electron microscopy.

The microtrench may be constructed in a manner to allow light to pass along the occupied channel, for example, so as to enable an assay utilising a planar waveguide approach for the purpose of an optical assay. Preferably a planar waveguide incorporated into the structure of the microtrench wall or walls would allow for lateral illumination of the cells within a microtrench, for example by light arising from a perpendicular aspect of the microtrench. This provides selective illumination of cells within the trench, or excitation of fluorogenic substances within the trench, the excitation being advantageously at 90 degrees to a preferred axis of observation of emitted fluorescence. The microtrench may be fabricated to incorporate surface and substrate properties that provide for optical sensing or excitation through the proximity of an adherent cell with the microtrench surfaces (e.g., plasmonic structures).

The array can be mounted in dishes, flasks and wells (e.g. 96 well plates) traditionally used to maintain cell viability, or on chips or microscope slides to aid viewing by microscopy. Alternatively the array may be an integral part of such cell culture vessels. These may be transparent to enable optical imaging, as discussed above. The advantage of incorporating the array into such standard equipment means that it is possible to analyse the array using equipment set up for receiving the standard equipment. Arrays having cells cultured within the microtrenches may be fixed by conventional means (e.g., 4% paraformaldehyde fixation), prior to processing for further analysis.

In the method according to the first aspect of the present invention patterns of cells within the one or more microtrenches can be deduced since analysis of microtrenches can be made at intervals after initial identification of microtrench occupancy. Analysis of the microtrench allows different types of cell behaviour to be identified by examining microtrenches at a single early time point after cell introduction, to determine initial occupancy, then at a later single time point measuring residual occupancy as determined by the nature of the cell type and the assay needs. This provides for a view of microtrench outcomes that map particular responses in terms of proliferation (gain in cell number), cell loss and proliferation arrest/delay.

The number of cells within a microtrench relates to proliferation/loss of cells by comparison at two different time points. In addition, the order of cells within a microtrench provides a read-out of divisional history and potential.

The microtrench can be used to identify cells that have not undergone proliferation or commenced proliferation upon the addition of a stimulus or other agent such as a test compound or cease division upon addition of a test compound enabling rapid assays for modulation of proliferation potential.

A characteristic of a single occupancy microtrench later reporting three cells is that one daughter of the progenitor must have undergone a further division, i.e., a progenitor has generated a surviving daughter that has undergone in excess of a complete cell cycle. This defines a measurement of minimum clonogenic potential (MCP), enabling a rapid assay for clonogenic potential of a progenitor. Prevention of cell/cell bypass within the microtrench dictates that the central cell of a three cell string is a MCP reporting cell, i.e., a cell which is itself the result of the division of a progenitor cell's daughter (and is, therefore, a second generation cell). Thus, a characteristic of a single occupancy microtrench reporting three cells is that a granddaughter (second generation cell) of the original single progenitor is now located in the centre of the progeny triplet. This enables the characteristics of an MCP cell to be examined (for example, by fluorescent protein signalling, immunochemical staining or immunofluorescence) as either a live cell or following fixation, since a cell which definitely displays MCP can readily be identified.

Comparison of a MCP cell with a single occupancy microtrench which, for example, continues to yield a single cell, allows for a comparison to be made between characteristics of an arrested/non-proliferating cell and those of an MCP cell. Such a comparison can be made to screen for the correlation between a given characteristic and the presence or absence of modulation of a specified target protein or cellular molecule as part of a drug discovery or evaluation process.

According to a second aspect of the invention, there is provided a method of determining the effect on cells of a given cell type of one or more test agents comprising the steps of allowing a first cell to adhere to a first microtrench, allowing a second cell to adhere to a second microtrench, exposing the first cell to the one or more test agents and determining whether the first and second cells exhibit different behaviours to one another. The microtrench is as defined above for the first aspect of the present invention.

The test agent may be one having a known or anticipated effect, such as those defined above.

Each one or more test agents may be applied to the surface of the first microtrench, or form part of the material of the first microtrench, prior to the first cell adhering to the first microtrench. Alternatively or additionally, each one or more test agent may be introduced into the first microtrench after the first cell has adhered to the microtrench. By way of a further alternative or addition, one or more test agents may be introduced into the interior of the first cell, either before or after the cell adheres to the first microtrench. This may be by means of microinjection, which is particularly facilitated by use of a microtrench, since the cells to be injected are necessarily aligned along the length of the trench, allowing easy alignment of the microinjection needle and the possibility of the convenient injection of multiple cells using a single needle. Furthermore, as the cells are aligned, it is easy to determine which cells have been microinjected. The need to use markers to mark cells that have been microinjected can therefore be avoided.

Microinjection of one or more cells within a nominated microtrench could be used to establish the degree of cell-cell communication, advantageously preserving the linear order of cells for analytical purposes. It is recognised that cell-cell communication could arise by the formation of a gap junction, or nexus, providing a junction or channel between certain animal cell types that allows different molecules and ions, mostly small intracellular signalling molecules (intracellular mediators), to pass freely between cells. The junction connects the cytoplasm of cells and can be functionally determined, for example, by the passage of fluorogenic material between adjoining cells within a microtrench or the delayed passage of such material as junctions form.

The microtrench provides an advantage during the processing of cells during an assay that requires a change of medium (e.g., to remove a reagent or an introduced candidate bioactive molecule) since cells resident within a microtrench will be subject to reduced hydrodynamic shear forces (in comparison with cells on an open planar surface) and therefore resist dislodgment according to the orientation of the trench with respect to the direction of fluidic flow.

The one or more cells used in the method of the first and second aspect of the present invention may be a tumour cell and, most preferably, is a human tumour cell. The methods of the present invention can then be used to test agents for their activity against tumour cells.

According to a third aspect of the invention, there is provided a cell culture microtrench being defined on or in a surface of a substrate, wherein the ratio of the width of the microtrench to the maximum length of the short axis of a cell type of interest is about 6 or less, the maximum length of the short axis of the cell type being measured when a cell is in attached or adherent mitotic states, and wherein the microtrench is less than 550 μm in length.

Alternatively, the third aspect of the present invention provides a cell culture microtrench being defined on or in a surface of a substrate, wherein the ratio of the cross-sectional area of the microtrench to the maximum cross-sectional area of a cell type of interest is about 2 or less, the cross-sectional area of the cell type being measured when a cell is in detached or suspended form, and wherein the microtrench is less than 550 μm in length.

Preferably the microtrench according to the third aspect of the present invention is between about 100 and 550 μm in length and most preferably about 500 μm in length. As indicated above, this length allows a single trench to be conveniently imaged at appropriate resolution using transmission in a single field of view. Microtrenches that are longer than 550 μm also have the disadvantage that interference from other cells contained within the trench may confuse any analysis. By restricting the length of the microtrench, such a risk is reduced. Each end of the trench is formed such that cell entry by motility-based immigration is prevented. For example, one or more ends of the trench may be defined by an end surface or wall or by the tapering of the microtrench to prevent the passage of a cell from an adjoining microtrench.

Other features of the microtrench according to the present invention are as defined above.

According to a fourth aspect of the present invention, there is provided an array comprising a plurality of the microtrenches according to the third aspect of the present invention. Other features of the array are as defined above.

According to a fifth aspect of the present invention, there is provided a method of manufacture of a microtrench according to the third aspect of the invention, or an array according to the fourth aspect of the invention. The material forming the microtrench may comprise an elastomeric material such as PDMS. Alternatives to PDMS include glass and SU8.

Preferably, the substrate is made of any material capable of being formed into a microtrenched condition such as glass, co-polymer or polymer, most preferably urethanes, rubber, molded plastic, polymethylmethacrylate (PMMA), polycarbonate, polytetrafluoroethylene (TEFLON™), polyvinylchloride (PVC), polydimethylsiloxane (PDMS), polysulfone and the like. Such substrates are readily manufactured from fabricated masters, using well known molding techniques, such as injection molding, embossing or stamping, or by polymerizing the polymeric precursor material within a mold. Standard soft lithography techniques may also be used to fabricate a substrate (for example: see Love et al. (July 2001) MRS Bulletin, 523-527; Delamarche et al. (1998) Journal of American Chemical Society 120 500-508; Delamarche et al. (1997) Science 276 779-781; Quake et al. (2000) Science 290 1536-1540; U.S. Pat. No. 6,090,251). Such substrate materials are preferred for their ease of manufacture, low cost and disposability, as well as their general inertness to most extreme reaction conditions. These materials may include treated surfaces, such as derivatized or coated surfaces, to enhance their utility for microtrenched substrates.

A particularly preferred substrate is a silicone elastomer polydimethylsiloxane (PDMS). Exemplary polydimethylsiloxane polymers include those sold under the trade mark Sylgard by Dow Chemical Co., Midland, Mich., and particularly Sylgard 182, Sylgard 184, and Sylgard 186.

Photodefinable epoxies such as SU-8 have been demonstrated to be biocompatible under appropriate conditions (for example, see: Ross et al. Micro Total Analysis System 2003, Squaw Valley, Calif., (2003) pp. 1061-1063) and there have been advances in the fabrication of three-dimensional structures based on photosensitive epoxies (Yoon et al. (2003) “Micromachined Polymeric Microvasculatures: A Three-Dimensional Microfluidic System Using Inclined SU-8 Structures and Laser Machining”, 226th American Chemical Society National Meeting) that would be suitable for microtrench fabrication.

An aspect of the substrate is that it should be amenable to typographic profiling and modulation of surface characteristics. Frequently used methods of modulating the attachment and growth of cells to a substrate, by way of non-limiting example of PDMS, include: changing the chemical composition of PDMS, surface treatment of PDMS with plasma, surface treatment of PDMS by coating with negatively charged molecules (e.g., poly-D-lysine, lecithin) and the deposition of extra-cellular matrix (ECM) proteins (e.g., laminin, fibronectin, collagen). Topographical and physicochemical modification of a biocompatible surface and the printing of cell-guidance and adhesion molecules onto a material have been described previously (Jung et al. (2001) Critical Reviews in Biotechnology 21 111-154). The low profiles of the microtrenches described in the invention act to aid the restricted deposition of, for example, adhesion molecules by physical means such as pin-based droplet delivery or high precision inkjet printing methods.

According to a sixth aspect of the invention, there is provided a method of selecting a cell which exhibits minimum clonogenic potential (MCP) comprising placing a first cell in a constrained environment such that, if the first cell undergoes more than one cell cycle division, substantially all daughter cells are arranged sequentially in a line, and identifying a daughter cell that undergoes a further cell cycle division as exhibiting MCP. Preferably, the constrained environment is a microtrench as described above with respect to the first aspect of the invention.

Advantageously, this method, incorporating the use of a constrained environment such that all daughter cells arising from the cell division(s) of a first progenitor cell are arranged linearly, in a similar way to peas in a pea pod, enables reliable identification of non-progenitor cells after a period of cell growth and division. The operating principle is that daughter cells remain side-by-side following cell division in the constrained environment. Logic dictates that any cell which is not located at one end of the line of cells must be the product of at least one cell division event. Therefore, as discussed above, such a cell may exhibits MCP and may be used for further study as to the characteristics of a cell having MCP.

According to a seventh aspect of the present invention there is provided a method of transferring one or more cells contained in a microtrench to a different surface, comprising: bringing the microtrench and the surface into contact so that the one or more cells are in contact with the surface for a sufficient time to allow transfer of the cells to the surface, and removing the surface with the cells attached.

The microtrench is preferably as defined above with respect to the first aspect of the present invention. The surface can be any suitable surface to which the cells can be transferred, including one coated with poly-L-lysine. Other surfaces that can be used will have qualities that impart preferential adherence or binding of the cellular material, such as surface charge (e.g., PDMS exposed to a plasma arc to make its surface hydrophilic), covalent binding through a chemical interaction with a surface, and non-covalent binding (e.g., a surface prepared with an immobilised antibody with affinity for epitopes presented on the surface of the cell to be transferred).

The cells may be fixed (e.g., using 4% paraformaldehyde) prior to the transfer.

The advantage of transferring the cells to a surface is that the surface can then be processed to further analyse the cells using standard techniques. For example protein expression, morphological characterisation and molecular characterisation can be performed. As the cells have been directly transferred the position and orientation of the cells is an exact copy of the cells position within the microtrench. This is advantageous as the position of individual cells can be easily determined.

Throughout this specification, where mention is made of a cell or cell type, it is preferred that the cell or cell type is a live eukaryotic cell. More preferably, the cells are selected from the following cell types:

Animal cells including human and mammalian cells derived as biopsy specimens (e.g., by fine needle aspirates), as tissue explants, as primary cultures (e.g., human skin fibroblasts), as transformed cell lines (e.g., SV40 transformed fibroblasts), as immortalized cell lines (e.g., cell lines immortalized with human telomerase reverse transcriptase [hTERT]), or as established tumour cell lines.

Human tumour cell lines including those representing specific sites and diseases of therapeutic, diagnostic and analytical interest, preferably those capable of demonstrating adherent growth on a substrate, for example: Brain Cancer, Bladder Cancer, Breast Cancer, Colon and Rectal Cancer, Endometrial Cancer, Kidney Cancer (Renal Cell), Leukaemia, Lung Cancer, Melanoma, Pancreatic Cancer, Prostate Cancer, Skin Cancer (Non-melanoma), Thyroid Cancer. Also, Human tumour cell lines routinely available for the purpose of drug screening methodologies such as those indicated in the US National Cancer Institute tumour cell line panel NCI-60 (ref: http://dtp.nci.nih.gov/docs/misc/common_files/cell_list.html):

| | NCI-60 | | NCI-60 | | NCI-60 |
|---|---|---|---|---|---|
| Cell Line | Panel Name | Cell Line | Panel Name | Cell Line | Panel Name |
| A549/ATCC | Non-Small Cell | U251 | CNS | TK-10 | Renal |
| | Lung | | | | |
| EKVX | Non-Small Cell | LOX IMVI | Melanoma | UO-31 | Renal |
| | Lung | | | | |
| HOP-62 | Non-Small Cell | MALME- | Melanoma | PC-3 | Prostate |
| | Lung | 3M | | | |
| HOP-92 | Non-Small Cell | M14 | Melanoma | DU-145 | Prostate |
| | Lung | | | | |
| NCI-H226 | Non-Small Cell | SK-MEL-2 | Melanoma | MCF7 | Breast |
| | Lung | | | | |
| NCI-H23 | Non-Small Cell | SK-MEL-28 | Melanoma | MDA-MB- | Breast |
| | Lung | | | 231/ATCC | |
| NCI-H322M | Non-Small Cell | SK-MEL-5 | Melanoma | HS 578T | Breast |
| | Lung | | | | |
| NCI-H460 | Non-Small Cell | UACC-257 | Melanoma | MDA-MB-435 | Breast |
| | Lung | | | | |
| NCI-H522 | Non-Small Cell | UACC-62 | Melanoma | BT-549 | Breast |
| | Lung | | | | |
| COLO 205 | Colon | IGR-OV1 | Ovarian | T-47D | Breast |
| HCC-2998 | Colon | OVCAR-3 | Ovarian | LXFL 529 | Non-Small Cell |
| | | | | | Lung |
| HCT-116 | Colon | OVCAR-4 | Ovarian | DMS 114 | Small Cell Lung |
| HCT-15 | Colon | OVCAR-5 | Ovarian | DLD-1 | Colon |
| HT29 | Colon | OVCAR-8 | Ovarian | KM20L2 | Colon |
| KM12 | Colon | SK-OV-3 | Ovarian | SNB-78 | CNS |
| SW-620 | Colon | 786-0 | Renal | XF 498 | CNS |
| SF-268 | CNS | A498 | Renal | RPMI-7951 | Melanoma |
| SF-295 | CNS | ACHN | Renal | M19-MEL | Melanoma |
| SF-539 | CNS | CAKI-1 | Renal | RXF-631 | Renal |
| SNB-19 | CNS | RXF 393 | Renal | SN12K1 | Renal |
| SNB-75 | CNS | SN12C | Renal | MDA-MB-468 | Breast |

Also, human tumour cell lines selected for their functional expression of specific molecular entities such as transporters of xenobiotic molecules (e.g., the ABCA3 drug transporter expressing in lung cancer lines H522M, A549, and EKVX) and human tumour cells selected for their convenient performance in gene transfer studies (e.g., U2-OS human osteosarcoma cells).

Mammalian cell lines used in functional genomics studies (e.g., NIH 3T3 murine cell line)

Single-cell forms of vertebrates (e.g., components of embryos, larval forms or cells derived from dissociated tissue preparations of zebrafish (Danio [Brachydanio] rerio).

Cell lines used in ADME/Tox (Absorption, Distribution, Metabolism, Elimination/Toxicity) screening protocols (e.g., hepatocyte derived cell lines such as HepG2).

Embryonic stem cells derived from human or murine sources.

Adult stem cells

Neurones and/or supporting cells of the central nervous system (e.g. astrocytes, oligodendrocytes, microglia and Schwann cells).

Immortal somatic cell hybrids including hybrids that secrete antibodies (e.g. hybridomas).

The skilled person will understand that the use of a microtrench or an array of microtrenches allows the identification and analysis of a diverse range of behaviours, including:
Different cell types in which they may vary according to cell division potential or behaviour.
Stem cell versus non-stem cell.
Senescent versus non-senescent.
Adherent versus non-adherent.
Quiescent versus non-quiescent.
Sensitive to proliferation stimulation versus refractory to stimulation.
Changes in cell behaviour on the micropatterned surface due to physiological changes in cells (e.g., differentiation or change in growth phase).
Changes in cell behaviour on the micropatterned surface due to changes in cells in response to a disease process:
   Those disease states induced by an infective agent including bacteria and viruses;
   Those disease states induced by a parasite.
Changes in cell behaviour on the micropatterned surface due to changes in cells in response to a physical agent (e.g., ionising and non-ionising radiations).
Changes in cell behaviour on the micropatterned surface due to the incorporation of optical active physical agents (e.g., quantum-well carrying nanoparticles) or chromatic dyes.
Changes in cell behaviour on the micropatterned surface due to changes in cells in response to an known or unknown bioactive agent for the purpose of:
   Environmental sensing (e.g., heavy metal contamination).
   Detection of nanoparticle (conveniently describing a particle having one or more dimensions of the order of 100 nm or less) toxicity with the advantage that cells carrying a toxic load of particles and undergoing proliferation arrest can be located for definitive analysis using electron microscopy or other high resolution imaging approaches.
   Detection of toxins (e.g., endotoxin sensing for bio-safety).
   Detection of toxic or harmful agents for security monitoring purposes and rapid diagnostics.
   Monitoring the progress of a fermentation process (e.g., yeast life cycle in a brewing application).
   Monitoring the progress of a biopharmaceutical preparation process (e.g., cytokine production).
To discern state transitions associated with cell death (apoptosis or necrosis).
Analysis of cell cycle progression in physiological and pathological systems.
Analysis of pharmacodynamic responses for the purpose of drug screening or discovery.
The study of cellular systems that modulate cell structure and function as they undergo state changes under the influence of internal programmes or enforced by perturbing agents (e.g., cytoskeleton or chromatin modulating agents).

### DESCRIPTION OF THE FIGURES

Embodiments of the invention will now be described, by way of example only, with reference to the accompanying Figures in which:

FIG. 1 shows, conceptually, how three variable influences (degrees of freedom) may be selected to describe conditions suitable for a cell-based assay on a microfeatured surface;

FIG. 2 shows the impact of substrate quality, representing an S variable shown in FIG. 1, on an exemplar cellular behaviour characteristic (motility);

FIG. 3 shows some options for trench profiles, as distinct from the separating walls for parallel sets of trenches, together with options for location with respect to the substrate plane;

FIG. 4 shows examples of the impact on varying microtrench dimensions on occupancy;

FIG. 5 shows an exemplar CAD pattern for an array of microtrenches;

FIG. 6 shows the appearance of different sets of fabricated microtrenches. The 500×500 μm dimensions of each set conveniently fits a field of view for conventional light microscopy;

FIG. 7 shows AFM (Atomic Force Microscopy) verification of the surface topology of an exemplar microtrench. AFM is a device used to image materials at the atomic level;

FIG. 8 shows the results of a typical microtrench array analysis to screen for the impact of microtrench dimensions on the motility and associated behaviour of adherent cells as a method of allowing a user to identify combinations of dimensions that impose advantageous constraints;

FIG. 9 is a scanning electron microscope image of a U-2 OS cell growing in an adherent form within a microtrench formed in PDMS, demonstrating cell alignment with the long axis of the microtrench;

FIG. 10 shows a typical analysis of the impact of TC plastic versus PDMS versus microtrenched PDMS on the orientation of mitotic division events;

FIG. 11 shows a diagrammatic representation of 9 different examples for microtrench assay outcomes;

FIG. 12 shows a comparison of cell division on TC plastic (upper panels) versus a microtrenched surface (lower panels) demonstrating the ordered arrangement of division products in a microtrench;

FIG. 13 shows typical readouts that can be derived from microtrench-based assays;

FIG. 14 demonstrates the clonogenic potential of human tumour cells (U-2 OS osteosarcoma) on both tissue culture plastic (left panels a, c, e and g) and PDMS (right panels b, d, f and h) determined at 8 days (a, b), 11 days (c, d) or 18 days (e-h) incubation. Images demonstrate the similar ability of the two surfaces to sustain the formation of colonies derived from single cells surviving exposure to UV radiation (5 Jm⁻²; 254 nm wavelength);

FIG. 15 demonstrates the clonogenic potential of human tumour cells (U-2 OS osteosarcoma) on both tissue culture plastic and PDMS, together with the similar ability to report relative reduction in clonogenic potential following UV irradiation (5 Jm⁻²; 254 nm wavelength);

FIG. 16 shows the transfer of cells from blank PDMS surface to poly-L-lysine slide surface (right panel). Giemsa staining reveals that no cells are left on the PDMS surface (left panel), the cells retain good morphology upon transfer. Both transmission images obtained at the same magnification; and

FIG. 17 shows the transfer of cells from a microgroove PDMS surface to poly-L-lysine-coated glass microscope slide surface. (a) Microtrench PDMS surface devoid of cells after conducting transfer process—donor surface. (b) Recipient surface, in this a case poly-L-lysine coating, with the cellular layer and stained cellular material apparent and a retention of an impression of microtrench features and their alignment. (c) same as b with regions of interest outlined to depict the location of nuclei.

### EXAMPLES

### Selection of Substrate and Topological Conditions to Influence Cell Adhesion

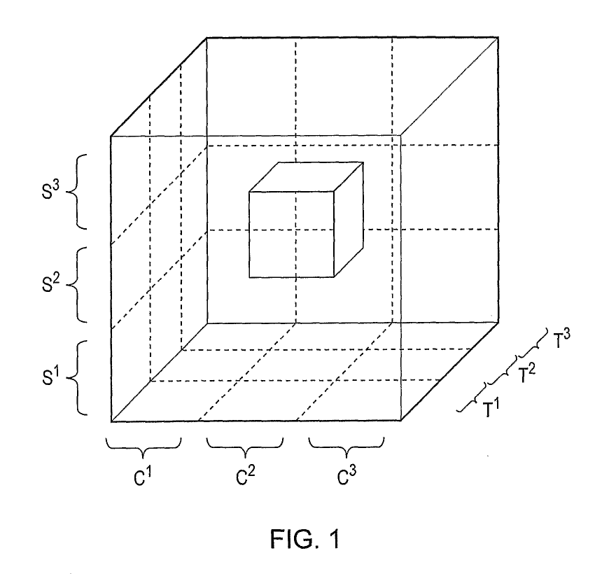

FIG. 1 shows conceptually, by way of example, how three variable influences (degrees of freedom) may be combined to describe conditions suitable for a cell-based assay on a microfeatured surface. The quality S represents substrate characteristics that can be varied according to different degrees S¹, S², S² or Sⁿ. The quality C represents cell-associated characteristics that can be varied or are presented according to different degrees C¹, C², C² or Cⁿ. The quality T represents topological characteristics of the substrate that can be varied according to different degrees T¹, T², T² or Tⁿ. By way of example, S′ could represent tissue culture (TC) plastic, S² an unmodified PDMS substrate and S³ a modified PDMS substrate. By way of example, C¹⁻ⁿ could represent a cellular characteristic such as the variable expression of a cell surface adhesion molecule. By way of example, T¹⁻ⁿ could represent the variable dimension of a microtrench such as its width or cross-sectional area. The central cube shown in the concept diagram thus represents the selection of a combination of characteristics (in this case S² and C² and T²), defined by a user that has selected a certain topological feature (e.g., T² being a microtrench with a ratio for trench width to cell short axis length of 0.5) to be appropriate for cells with a given characteristic (e.g., C² being the ability to show changed motility on a specific substrate) demanded within an assay on a given substrate of with certain characteristics (e.g., S² being an unmodified PDMS substrate).

### Modulation of Cell Motility by Substrate Selection

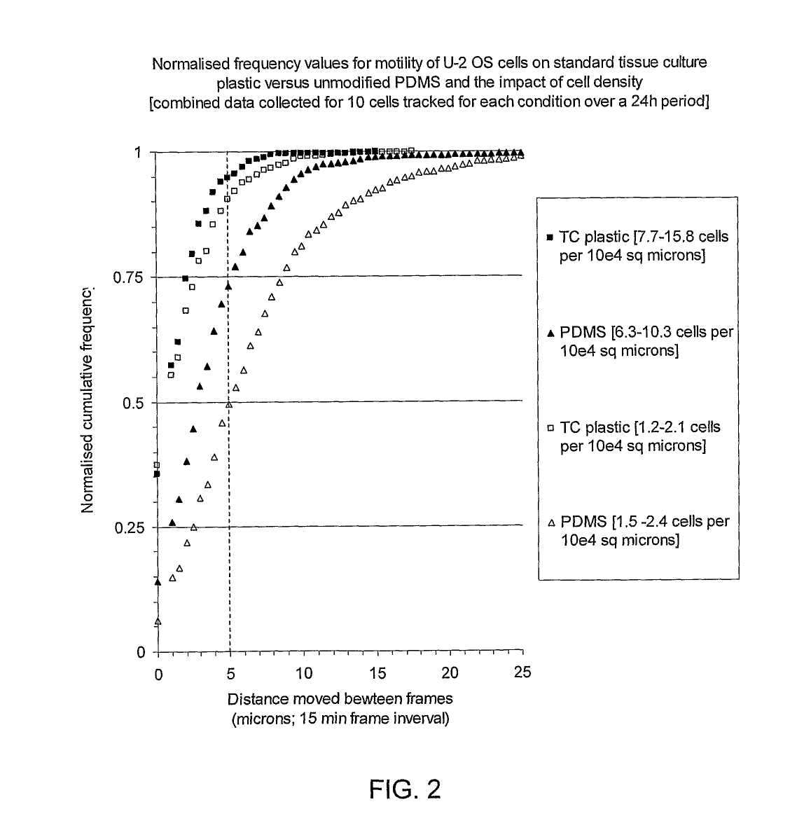

FIG. 2 shows the impact of substrate quality, representing an S variable shown in FIG. 1 on an exemplar cellular behaviour characteristic (motility). U-2 OS cells (ATCC HTB-96) were cultured under normal conditions on tissue culture grade plastic surfaces (Smith et al. (2007) Cell Cycle 6 2071-81) prior to detachment by brief enzymatic digestion, counting and over-layering onto TC plastic (the base of a well within a standard 6-well plate) or unmodified PDMS (a layer attached to the base of a well within a standard 6 well plate) in fresh culture medium and incubation at 37° C. and 5% CO₂ in air to allow for the attachment of cells and their subsequent proliferation. Cells were observed and recorded using time-lapse microscopy as described previously (Marquez et al. (2003) Oncogene 22 7642-8). MetaMorph software (Molecular Devices, California) was used to view the stacked image as sequences of images to assess cell movement between frames and therefore motility. The data are normalised frequency values for motility of U-2 OS cells on standard tissue culture (TC) plastic versus unmodified PDMS and the impact of prevailing cell density. Images of a fixed view for a given substrate were acquired at 15 min frame intervals over a period of 84.25 h for cell cultures undertaking adherent growth under standard cell culture conditions.

FIG. 2 shows the results of combined data collected for 10 independent cells tracked for each condition over an approximate incubation period of 24 h at the start or the end of the imaging period. The data show that overall motility is enhanced on PDMS surfaces. Fewer cells show zero distance moved between consecutive frames on PDMS (6.3-14.0% total events) compared with TC plastic (35.6-37.3% total events). Further, motility decreases on both substrates as cell density increases. This demonstrates that local density is capable of impeding cell movement introducing a variable into an assay system that does not attempt to constrain this influence. Irrespective of prevailing cell density, greater motility is demonstrated on PDMS, as demonstrated at the 0.5 fraction level (i.e., 50% frequency), at which the distance traveled on PDMS is 2.5-fold greater than the distance traveled on TC plastic. The analysis shows that cells with adherent properties may be capable of successful attachment and spreading and enhanced motility on a relatively hydrophobic substrate imparting a degree of plasticity to cellular behaviour, such that topological features (e.g., microfeatures such as microtrench pitch) can then be used to potentially direct the forms of cell attachment, adherence and alignment.

### Microtrench Profiles and Limitations on Occupancy

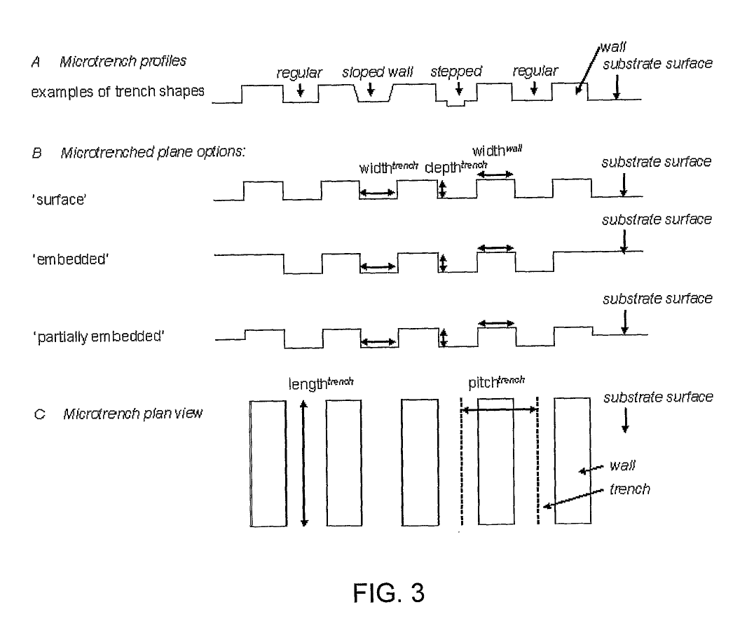

FIG. 3 shows some of the options for trench profiles, as distinct from the separating walls for parallel sets of trenches, together with the options for location with respect to the substrate plane; the figure also shows the basic trench-associated dimensions for reference, the aspect ratio being the ratio of the given dimension to the equivalent dimension of the cell.

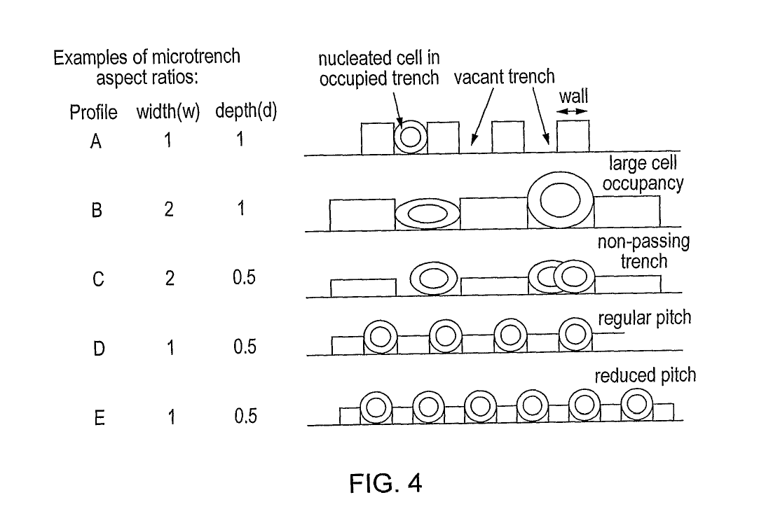

FIG. 4 shows examples of the impact on varying microtrench dimensions on occupancy; the microtrenches may have variable lengths within an array but always with a long axis (l) greater than the width (w).

### Arrays of Microtrenches, Fabrication and Examination

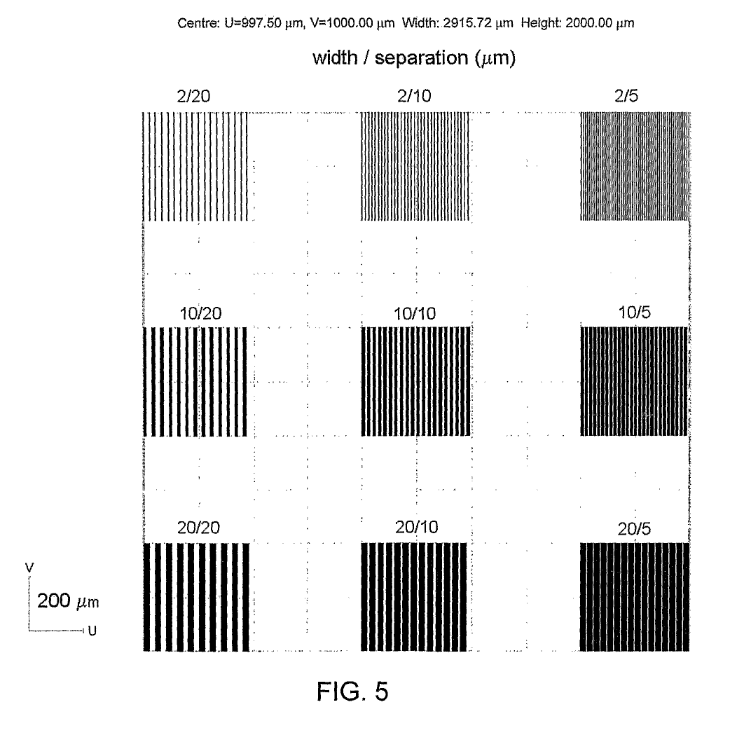

FIG. 5 Exemplar CAD pattern (CAD package Autosketch) for an array of microtrenches. The Figure shows nine different microtrench types in which the width and separation of trenches has been varied. In the design shown, the microtrenched areas are also separated by non-trenched areas to aid the analysis of the impact of the substrate alone without any microfeatures. These areas could also carry encoding information for a given array. By way of example, the area designated 20/20 would be used to generate nine trenches, the lower surface of which is contiguous with the non-trenched areas.

Microtrenches can be prepared using known methods and using customised arrays of various patterns of microtrenches, for example having different widths and/or inter-trench spacing. A typical method for preparing such an array is described briefly. Glass substrates are spin coated (to a thickness of between 0.5 and 1.5 um) with a positive photoresist. (Shipley resists 1805 or 1818). Designs from a CAD package (Autosketch) are exported to a direct-write mask-writing machine (DWL66, Heidleberg Instruments). The designs are then serially written into the resists, which were subsequently developed (30 s in MF319, Chestech). A standard mix of PDMS (10:1) is then poured onto the glass substrates with photoresist patterns. After curing on a hotplate for 30 mins at 70° C. the PDMS copy can be peeled off. The design and fabrication method can be used to generate microtrench features covering a parameter space between ˜1 μm and 20 μm.

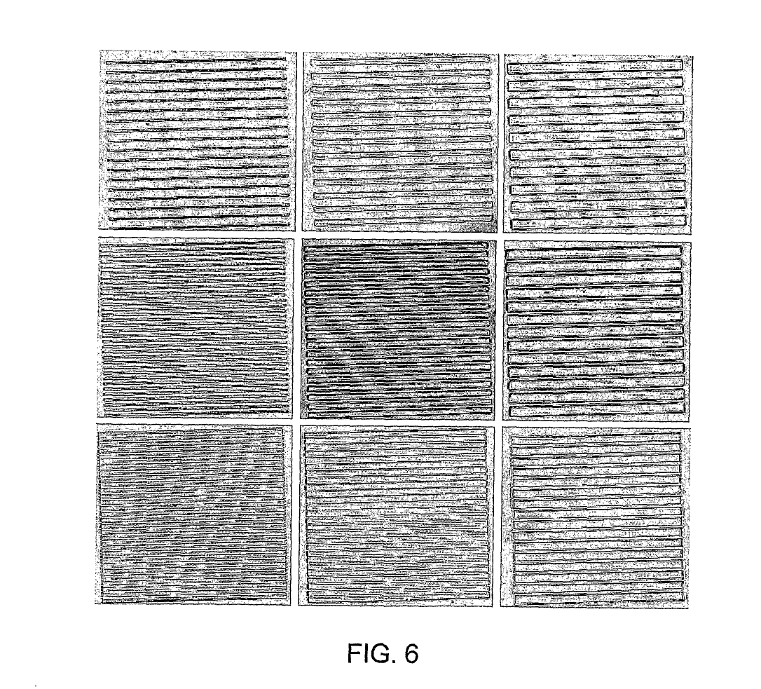

FIG. 6 shows the appearance of different microtrench dimensions within a fixed area convenient for examination by conventional light microscopy.

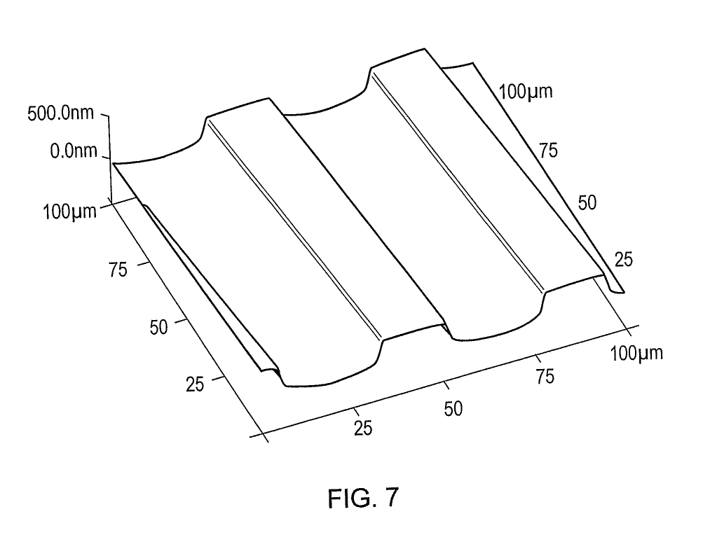

FIG. 7 shows AFM surface rendition of a typical low profile microtrench (designed 16/30 dimensions). AFM (atomic force microscopy; system Vecco AFM) conventionally uses a ceramic or semiconductor tip positioned at the end of a cantilevered bar. As the tip is moved over the material, it either continuously touches or periodically taps the surface and bends as it is repelled or attracted to the structure with the linked deflections being detected and converted into distance measurements. AFM can be used to image non-conductive materials. Using AFM the 16/30 microtrench was found to have a nominal width of 27.36 μm, with adjacent microtrenches being separated by walls which were nominally 16.71 μm thick. The analysis confirms the translation of design to real physical features using the exemplar fabrication method described.

### User Selection of Optimal Microtrench Dimensions for Constraining Cell Orientation by Growth in Microtrenches

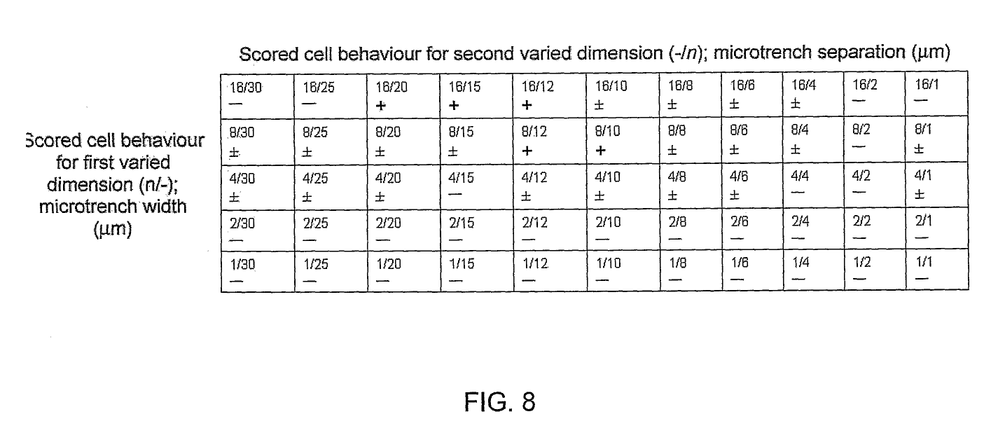

FIG. 8 shows a typical microtrench array analysis to screen for the impact of microtrench dimensions on the motility and associated behaviour of adherent cells as a method of allowing a user to identify dimensions that impose advantageous constraints. A typical microtrench array is demonstrated, using, for example, U-2 OS human osteosarcoma cells (nuclear diameter approx 12 μm). FIG. 8 shows an 11 by 5 array format representing a range of combinations for trench width versus separation (i.e. a code of 16/15 indicates a width of 16 μm and a separation of 15 μm). Thus, by way of example, width is varied according to columns and the distance between trenches is varied according to each row. The PDMS surface array containing microtrench formats used a fixed height (d) of 1.8 μm, although this third dimension could be easily varied to advantage. Each microtrenched area (x by y) was a minimum of 300 μm by 300 μm. The PDMS arrays were fabricated in a large block 100 mm×100 mm and cut into a disk to fit tightly in a 24 well coverslip bottomed multi-well plate. U-2 OS ATCC cells were seeded onto the structures and left overnight at 37° C. All original cell concentrations were determined using a Coulter Particle Counter (Beckman Coulter, High Wycombe, UK). The plate was then placed onto a microscope stage with an incorporated incubator. The instrument comprised a Zeiss Axiovert 100 microscope (Zeiss, Welwyn Garden City, UK) fitted with a temperature regulating incubator system and CO₂ supply (Solent Scientific, Portsmouth, UK). The motorised xy microscope stage was from Prior Scientific and the phase transmission images (x10 objective lens) were captured every 15 min over 24 h (144 frames per field) or followed by a further 24 h using an Orca I ER charge-coupled device camera (Hammamatsu, Welwyn Garden City, UK). The camera, stage (xy) and focus (z) were PC controlled via AQM 2000 software (Kinetic Imaging, Wirral, UK). Each position of the microtrench array was sampled every 15 minutes. Tiff-format Images (512×512 pixels) were played back for analysis as movies using MetaMorph software (Molecular Devices Corporation, PA, USA). The timelapse image sequences were observed to determine the impact of the microstructures on cell movement (movement parallel to microtrench orientation), cell division (rate, orientation), and cross-talk (movement between trenches) and the ability of cells to pass in a trench.

FIG. 8 shows the scores for the following evaluations:

−: indicates zero or minimal constraint on cell movement;
±: indicates some constraining associated with microtrenches;
+: indicates preferable microtrench dimensions and configuration for U-2 OS cells to constrain movement parallel to the microtrench, including no cell passing within the trench and minimal cross-talk between trenches. According to the method described a user would preferably select microtrench dimensions of 16/15 for use with U-2 OS cells for use, for example, in an assay for MCP.

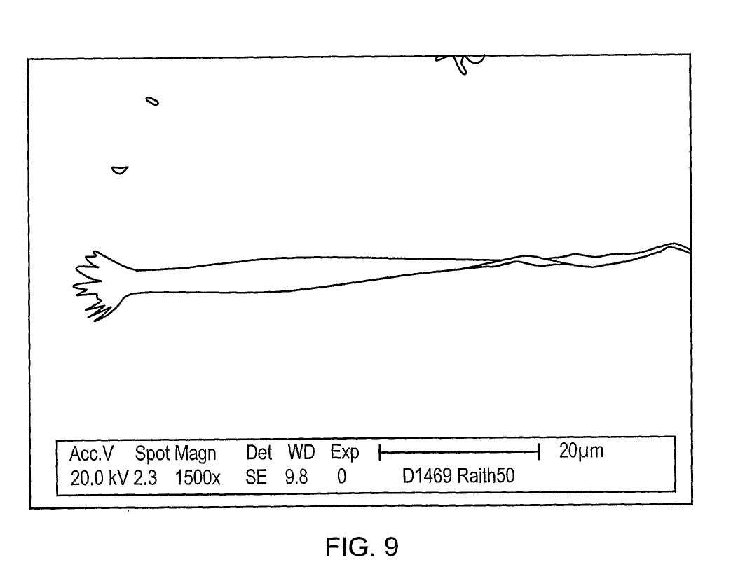

FIG. 9 shows a scanning electron microscope (SEM) image of a cell growing within a microtrench. Indicating that the cell extends along the longitudinal axis of the trench and does not tend to extend “outside” the trench, i.e., to extend up the sides of the microtrench or even to extend into an adjacent trench. A typical protocol to obtain cell attachment to microtrenches is described here.

Standard cell culture methods, as described in conjunction with FIG. 2 above, can be used to overlayer a cell suspension (e.g. of the human osteosarcoma cell line U-2 OS [ATCC HTB-96]). The cell suspension in fresh medium is allowed to settle under gravity onto the microtrenched surface (e.g., a layer of PDMS carrying a microtrench array pattern). The cells were maintained at 37° C. and 5% CO₂ using standard tissue culture techniques. Media used was McCoys 5A modified (Sigma) supplemented with 2 mM glutamine, 100 units/ml penicillin, 100 microg/ml streptomycin, 10% fetal calf serum and 1000 microg/ml geneticin. Immediately post-treatment, the cultured dishes were placed onto a timelapse instrument designed to capture transmission phase images from multi-well plates. An Axiovert 100 microscope (Carl Zeiss, Welwyn Garden City, UK) was fitted with an incubator for 37° C./5% CO₂ maintenance (Solent Scientific, Portsmouth, UK), and an ORCA-ER CCD camera (Hamamatsu, Reading, UK). All images were collected with a x10 (PH1) apochromat objective lens providing a field size of 500×500 μm². Sequences were captured over 48 hours, sampling at 15 min. At the end of an experiment the images were stacked and saved. MetaMorph software (Molecular Devices, California) was used to view the stacked images as sequences of images and analyse cell behaviour of interest. It was found that, when a culture of adherent cells was applied to such arrays, some arrangements of microtrenches resulted in cells preferentially adhering to the surface of the microtrenches, in preference to any other surface within the array. It was determined that, where the ratio of the width of the trench to the nominal diameter of the cell, determined in a suspended form was less than 2, the cells would exhibit the behaviour of preferentially locating and aligning themselves within the microtrench.

### Constraining the Orientation of the Axis of Cell Division by Growth in Microtrenches

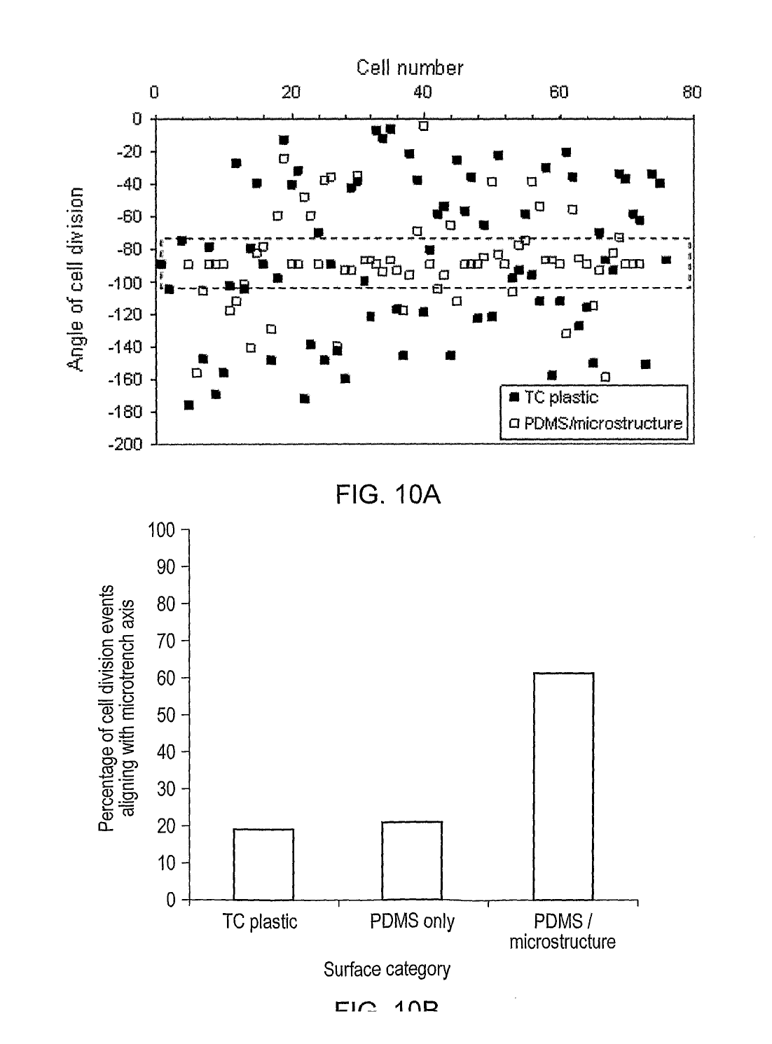

FIG. 10 shows a typical analysis, using routines available in conventional packages (MetaMorph software; Molecular Devices, California), of the impact of TC plastic versus PDMS versus microtrenched PDMS on the orientation of mitotic division events (the axis along which daughter cells separate during cytokinesis. Individual mitotic events were analysed in multiple fields and the angle of cell division determined for each substrate. In the example shown the spread of data is shown in panel a for TC plastic versus PDMS/microstructure allowing an orientation consensus to be defined (−90° represent in alignment with the long axis of the microtrench; −105° lower limit for orientation; −75° upper limit for orientation; 177° indicates perpendicular to trench axis). Using this consensus, the mean data are shown in panel b clearly indicated the enhanced ability of a microtrenched surface to impose orientation on the axis of cell division. This demonstrates the ability of a microtrench to orientate and thereby constrain the products of cell division to 1 dimension.

### Assay of Progenitor Cell Behaviour and Fate Using Microtrenches

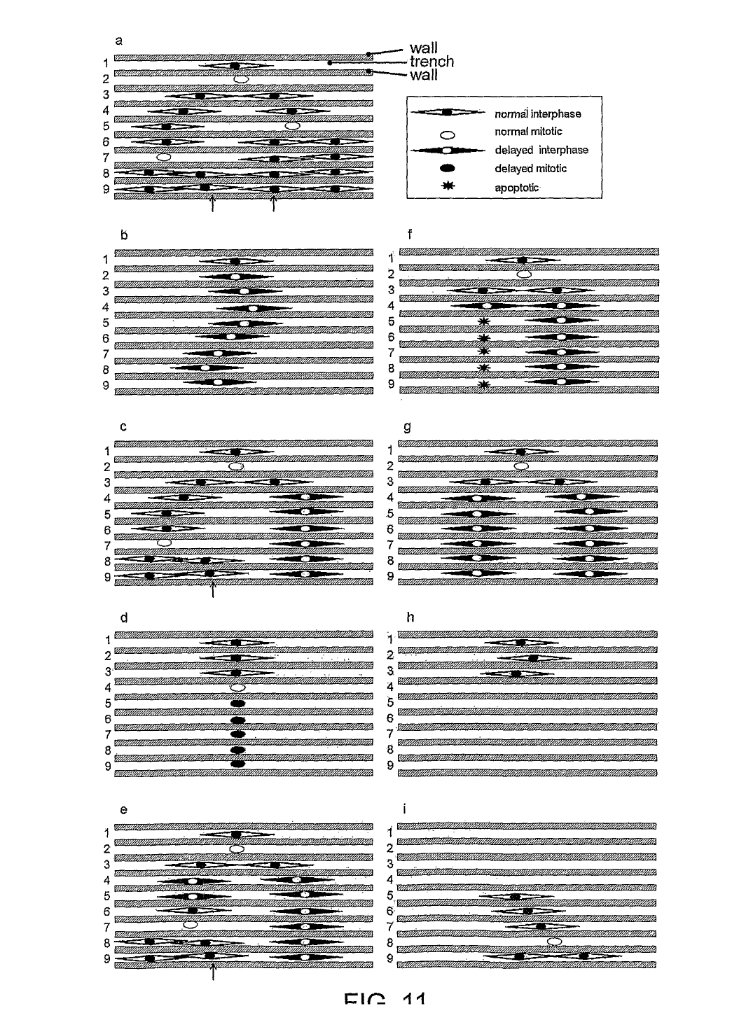

FIG. 11 shows a diagrammatic representation of 9 different microtrench assay outcomes in which the single trenches a-i are also represented at different arbitrary time intervals numbered 1-10. Thus, for example, a1, a2 to a9 represent the same single trench visualised at increasing time points and in which a single progenitor cell, observed at time point 1 and 2 proliferates to generate four descendants observable at time points 8 and 9. The quality of cell events and states are shown in the inset key.

FIG. 11 shows examples of possible outcomes, as the result of:
a: clonogenic progenitor and asynchronous daughter generating 4 second-generation cells,
b: arrested/delayed progenitor cell;
c: asymmetric cell cycle arrest/delay of mitotic products;
d: extended mitosis;
e: asymmetric recovery of delayed mitotic products;
f: daughters showing death and delay;
g: symmetrical arrest/delay of daughter cells;
h: progenitor loss by time point 4;
i: incursion into an unoccupied microtrench observed at time point 5.

In FIG. 11, cells marked with an arrow “⇓” are cells which have minimal clonogenic potential, i.e., they have been shown to undergo more than one complete cell cycle, including at least two generations from the progenitor and are occupying a trench position that allows for unambiguous designation of that divisional history.

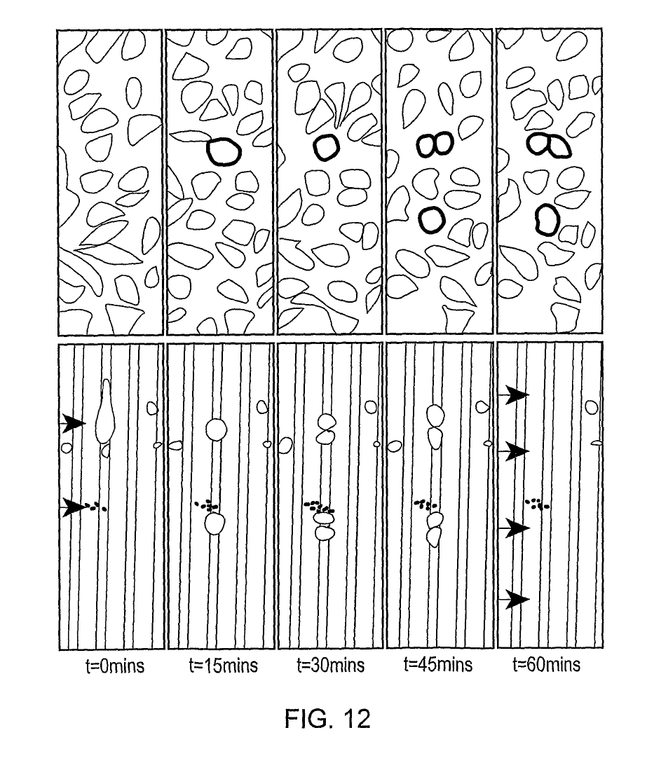

FIG. 12 shows a comparison of cell division on TC plastic (upper panels) versus a microtrenched surface (lower panels) in which an alignment of cell division in the latter allows for the simple tracking of cell division and the origin of descendants. In the microtrench example the length of the microtrench is clearly sufficient to record four cells arising from two progenitors. For example, taking the 0 and 60 min time points the order of progeny is maintained. In this case the inner two cells arrowed at 60 mins arise from different progenitors, observed at 0 mins.

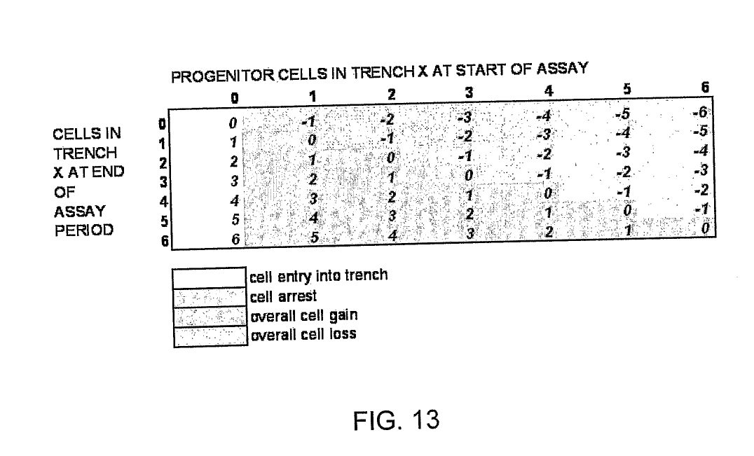

FIG. 13 shows typical readouts that can be derived from microtrench-based assays to provide simple determinations of cell arrest, cell gain and cell loss. The Figure shows a simple outcome with a microtrench (X) initially bearing a single progenitor providing highly informative information according to the time interval between the two observations.

### Microtrench-Enhanced Methods of Cell Analysis

As outlined above, a microtrench initially occupied by a single cell and reporting a cell at a later time point identifies a cell with a compromised proliferation potential. Such slowly proliferating or cell cycle arrested cells may be a minor component of a population and their relative frequency reduced by ongoing proliferation of other fractions, resulting in problems of identification. The invention overcomes this problem resulting from response heterogeneity.

An array of microtrenches provides a structure by which cell position can be more easily determined by automated image analysis software. The patterned surface itself could easily be segmented from the images (e.g., by edge detection, Hough transform, texture/intensity classification). The cells themselves will lie in lines along the trenches. Segmentation of these periodic objects can be performed on this essentially one-dimensional signal.

The microtrenches restrict the morphology of the cells such that they can be more easily detected, located, segmented and counted by automated image analysis software when in colonies. The restriction of the cytoplasm to preferentially extend within the trench adds some predictable structure to the cell's appearance; the variability in cell appearance is the major stumbling block to many attempts to automate cytology.

Changes in morphology are constrained such that they do not disrupt the cell state

(interphase, mitotic, apoptotic), enabling recognition by automated image analysis software. Classification of the cell state is usually most successfully done with fluorescence imaging of a DNA marker. The DNA replication ability of the cell should not be disturbed and the skilled person will understand the means by which these methods can readily be applied.

Different microscopical imaging methods may be used, such as bright-field, fluorescence and phase contrast, or combinations thereof, for micro-structure and cell location as well as cell and cell-state classification. It could be envisaged that, for instance, phase contrast microscopy is used to detect and locate the microstructure of the patterned surface, bright-field microscopy is used to determine the cytoplasm extent and fluorescence microscopy used to determine nuclear (or other organelle) position and cell state.

The constraint of cell motility to a single dimension allows the use of automated image analysis software, such that motility can be measured with sparse time-lapse images. Cell motility measurements traditionally require imaging at short time intervals such that the cells can be tracked. This involves cell detection followed by cell correspondence where cells at one time point are matched with those at the next, which poses a significant problem. The severity of this problem is greatly reduced through the use of the trench pattern surface where cells are constrained to move in a single dimension, are unlikely to cross to neighbouring trenches, or to pass each other within a trench. This relaxes the requirement for regular imaging, as correspondence is easier.

### Method of Determining the Effect of an External Agent on Cell Behaviour

A variety of screening methods to quantify the effect of a given agent on cell survival, growth and proliferation, for example, of human tumour cell populations in vitro have been described. The general conventional method for the determination of clonogenic potential has been described (Franken et al. (2006) Nat. Protoc. 1 2315-9). These authors describe how a clonogenic assay or colony formation assay is an in vitro cell survival assay based on the ability of a single cell to grow into a colony. The colony is commonly defined to consist of at least 50 cells to permit macroscopic detection. The assay essentially tests every cell in the population for its ability to undergo “unlimited” division. Clonogenic assay is the method of choice to determine cell reproductive death after treatment with, for example, ionizing radiation, but can also be used to determine the effectiveness of other cytotoxic agents including drugs as part of a screening process. Often only a small fraction of seeded cells retains the capacity to produce colonies. Before or after treatment, cells are seeded out in appropriate dilutions to form colonies in 1-3 weeks. Colonies are fixed with glutaraldehyde (6.0% v/v), stained with crystal violet (0.5% w/v) and counted using a stereomicroscope. Various mathematical methods can be applied for the analysis of dose-response relationships. A microtrench-based assay would comprise similar standard methods for cell preparation, determination of cell density prior to plating, the introduction of known cell concentration to a suitable culture vessel with the surface having areas with microtrench patterns. A simple form of assay would be to allow cell attachment by gravity sedimentation of cells onto the surface and a sufficient period of incubation to allow for the spreading of cells within microtrench locations. After such a period of attachment (e.g., 8-12 h for U-2 OS cells) a microtrench clonogenic assay to determine MCP could be performed by observation of microtrench occupancy and cell state at two discrete time points. The first time point is to establish occupancy before, for example, a drug treatment and therefore the microtrench system should be conveniently accessible to optical examination. The second time point would be, for example, after a period of recovery from an acute exposure to a drug or after a period of continuous exposure to a drug. The second time point would advantageously be after a sufficient period had elapsed for cells, on average, to have progressed through one or two generations (e.g. approximately 24 h and 48 h respectively for U-2 OS cells). Interspersed observations between the two time points would advantageously enhance the quality of data available and provide finer analysis of, for example, the period of delay before division of a progenitor cell, a feature that could represent a pharmacodynamic read-out of interest.

Assessment of microtrench occupancy and state can be reported in an assay outcome matrix as shown in FIG. 13 and the frequency of arrested cells reported, or the frequency of progenitor cells showing MCP or long term arrest reported. Relating, for example, the dose of an agent to a microtrench analysis such a loss of MCP would be a typical area of interest. Alternatively the microtrench can effectively replace the requirement for time-lapse imaging in some applications such as the identification of cells that have failed to proliferate following a drug exposure (i.e., long term arrested cells). Additional analyses can be carried out on the cells either as live cell functional assays such as a beta-galactosidase activity measurement for cell arrest/senescence (Maier et al. (2007) Ann N Y Acad. Sci. 1100 323-32), or following fixation to preserve aspects of cell structure and macromolecule expression using, for example, conventional methods for immunofluorescence detection.

Demonstration of the Clonogenic Potential of Human Tumour Cells (U-2 OS Osteosarcoma) on Both Tissue Culture Plastic and PDMS and the Similar Reporting of Reduction in Clonogenic Potential Following Exposure to a Standard Cytotoxic Agent (Short Wavelength UV Light)

For comparative purposes two different U-2 OS cell lines were used: i) the reference cell line from the American Type Culture Collection (ATCC; Manassas, USA; ATCC Number: HTB-96) and designated ATCC U-2 OS and ii) a derived cell line expressing a G2M Cell Cycle Phase Marker EGFP construct and designated U-2 OS G2M (GE Healthcare). Recommended conditions and procedure were used for cell culture as described, except that culture medium for U-2 OS G2M was additionally supplemented with 1000 μg/ml geneticin. Cells were plated into standard 6-well tissue culture plastic plates (with or without a surface layer of PDMS) at 2000 cell/well. Following 24 h incubation cells cultures were washed gently with PBS and exposed to short wavelength UV radiation (predominantly 254 nm wavelength emitted from a low-pressure mercury vapour germicidal lamp), media replaced and incubation for clonogenic potential continued. Cultures were fixed and stained with Giemsa stain using standard methods and transmission images obtained by standard microscopy methods. Colony frequency was determined to calculate percent absolute clonogenicity (100×[colony count per well/cells plated per well]) or percent relative clonogenicity (100×[absolute clonogenicity treated/absolute clonogenicity control]).

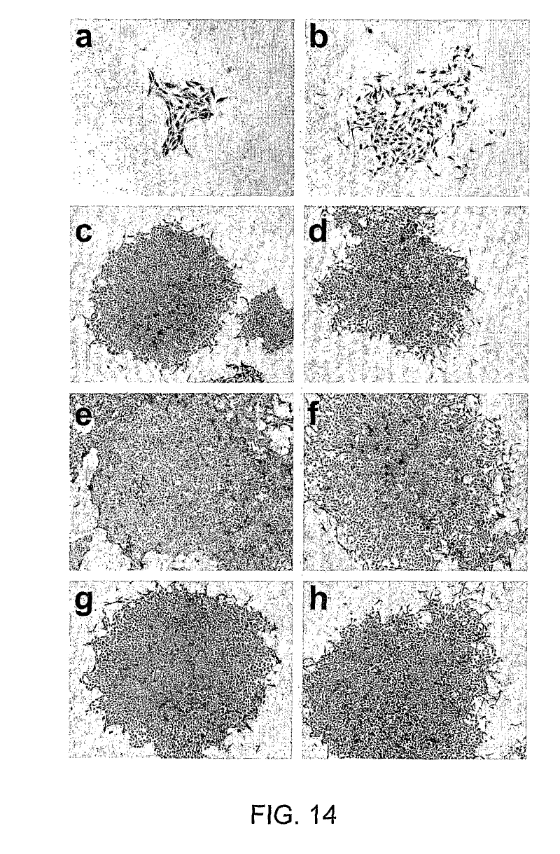

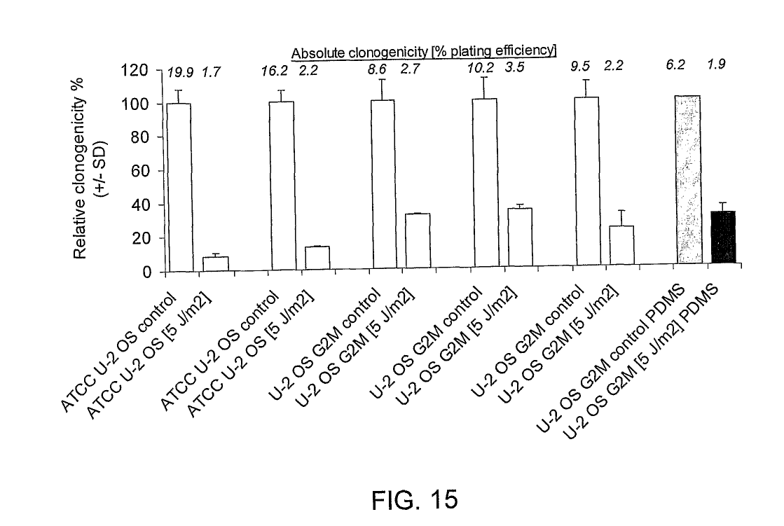

The results show that cultures can show similar clonogenic potential whether assayed on tissue culture plastic or on an unmodified PDMS surface (see FIG. 14) and that growth of cells on the PDMS surface does not compromise the ability to report relative clonogenicity in response to exposure to a cytotoxic agent (see FIG. 15)

Demonstration of Typical Method for the Extraction of Cell Proliferation Parameters (e.g., Intermitotic Time; IMT) from Cell Lineages Including Those Derived from Drug-Treated Populations and Those Constrained within a Microtrench

Human osteosarcoma cell line (U-2 OS (ATCC HTB-96)23) derived from a 15 year old Caucasian female were used. The cells were maintained at 37° C. and 5% CO₂ using standard tissue culture techniques. Media used was McCoys 5A modified (Sigma) supplemented with 2 mM glutamine, 100 units/ml penicillin, 100 μg/ml streptomycin and 10% fetal calf serum. The cells were seeded onto tissue culture plastic, PDMS layer with and without PDMS microtrench arrays and placed in a six-well multi-well dish (10⁴ cells per ml) and left to establish for 24 hours. Post-24 hours incubation the culture dish was then immediately placed onto a timelapse imaging instrument designed to capture transmission phase images from multi-well plates. An Axiovert 100 microscope (Carl Zeiss, Welwyn Garden City, UK) was fitted with an incubator for 37° C./5% CO₂ maintenance (Solent Scientific, Portsmouth, UK), and an ORCA-ER CCD camera (Hamamatsu, Reading, UK). All images were collected with a x10 (PH1) apochromat objective lens providing a full view of each microtrench format. Sequences were captured sampling at 15 min, and for three fields per treatment regime (using AQM, Kinetic Imaging, Nottingham, UK). MetaMorph software (Molecular Devices, California) was used to view the stacked image in sequence as for each experiment the images were stacked and saved as *.stk format.

Cells grown on PDMS surfaces did not present any special issues for imaging, the PDMS layer being sufficiently thin to enable access with a basic light microscope set-up so that cells could be observed and recorded growing on the substrate. Hence the behaviour on each surface was acquired and a simple analysis performed to determine the time between successive mitoses. IMT1 refers to the time between the division event generating the mother cell and that of the subsequent division generating two daughters, while IMT2 refers to the time between the division event generating the two daughters and the interval between a given daughter cell and the event generating granddaughter progeny (see tables 1 and 2).

TABLE 1
Determination of sequential IMTs (IMT1 and IMT2) from timelapse
imaging data to demonstrate the imposition of cell cycle delay apparent
in average IMT values for lineages arising from progenitors exposed to
a 1 h drug treatment (in this case the cytotoxic topoisomerase
I inhibitor topotecan)

| Drug treatment | IMT1 (av ± sd) | IMT2 (av ± sd) |
|---|---|---|
| Control | 21.1 ± 5.37 | 19.78 ± 4.9 |
| +10 μM topotecan (× 1 h) | 24 ± 4.4 | 22.9 ± 5.5 |

TABLE 2
Determination of sequential IMT from timelapse imaging (IMT1 and
IMT2) to show the maintenance of lineage integrity and cell cycle
progress on fabricated microtrench surfaces.

| Surface | IMT1 (av ± std) | IMT2 (av ± std) |
|---|---|---|
| Tissue culture plastic | 22.3 ± 1.6 | 21.4 ± 7.6 |
| PDMS flat surface* | 21.5 ± 9.4 | 23.1 ± 12 |
| Microtrench surface (16/12 format) | 15 ± 2.5 | 21.2 ± 1.7 |

*cells undergoing cell division extend the mitotic event by 1.5 to 2 times

The results show the relevance of intermitotic time (IMT) defined as the time interval between sequential mitoses of cells proliferating in a lineage. IMT analysis can describe the ability of a cell to traverse the cell cycle for a proliferating cell population thereby revealing cell cycle perturbations and delays in sequential generation. Cell cycle arrest or delay is a commonly used monitor of, for example, pharmacodynamic response. Time lapse microscopy is the repeated collection of a single field of view from a microscope at discrete time intervals through which dynamics of cell division can be captured.

Timelapse microscopy can be used for the tracking of single cell events or cellular responses to a bioactive drug. Transmission phase offers a probeless and non-perturbing contrast mode providing low resolution but highly informative outputs on cell behaviour (e.g., cell shape and cell position), the changes in these two basic features facilitate assays describing critical global cell cycle responses such as delay in cell division and other responses such as the induction of cell death and the modification of cell motility.

The results also demonstrate the similar extraction of IMT values from cells grown on three different surfaces (Table 2), including cells within microtrenches.

Typical Method to Demonstrate Transfer from a Microtrenched PDMS Surface to an Alternative Surface (eg a Coated Glass Microscope Slide)

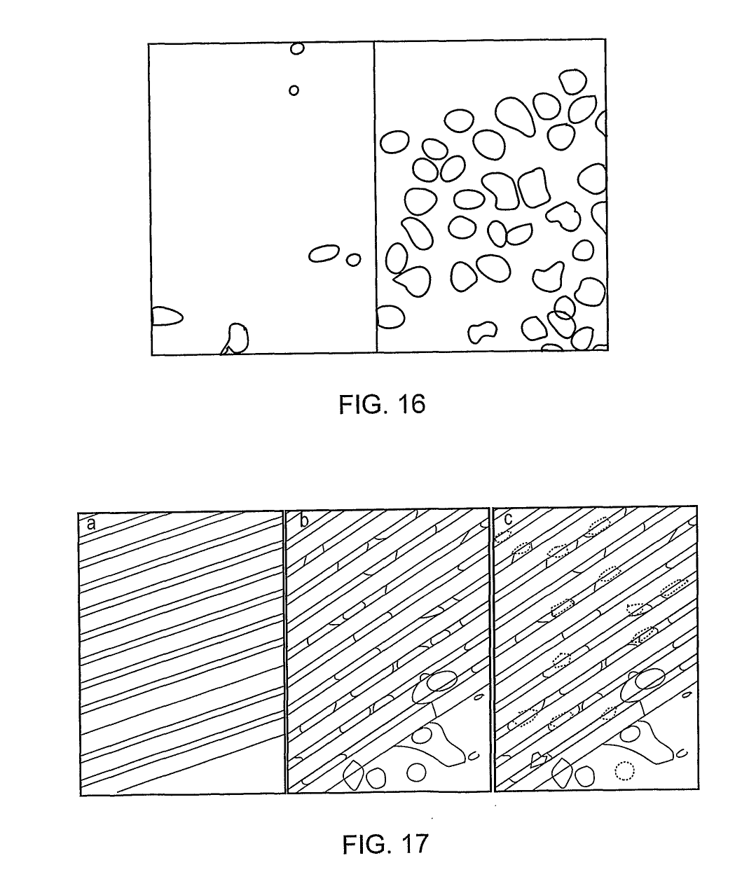

U-2 OS ATCC cells grown on PDMS layers with microtrench arrays (see FIG. 16) were fixed in 4% paraformaldehyde and subsequently washed in phosphate buffered saline and stored at 4° C. The cells can be stored from hours to months. To conduct the transfer all buffer was aspirated and the PDMS layer washed with water. The PDMS layer was released from the multiwell dish. Using sterile forceps the PDMS layer was placed onto a Poly-L-lysine coated microscope slide—with the cellular layer in direct contact with the poly-1-lysine surface. Excess liquid around edges was blotted with tissue and the sandwiched slide was left to dry (1-6 days).

Cell morphology (nucleus and cytoplasm) was determined using Giemsa staining (10%) for 20 minutes the PDMS layer and microscope slide was maintained sandwiched together. The PDMS layer was then peeled away to leave the cellular layer incorporating an impression of microtrench features on the Poly-1-lysine surface. Both the PDMS layer and the cellular layer were stained.

The results demonstrate a simple protocol for the transfer the cell samples from a PDMS surface to another (e.g., a standard glass microscope slide). The reduced adhesion of cells to PDMS or similar surfaces, even when microtrenched, provides for an opportunity to transfer cells to a second surface by mechanical pressing if the second surface has suitable adhesive properties (see FIG. 16).

It is possible to transfer by effectively printing the cells from a microtrench to an alternative surface. This allows for retention of orientation and relationships between cells for MCP analysis with respect to microtrench location (see FIG. 17). Such an effective transfer of cell location enables further analysis of the cells, e.g., investigation of other properties of those cells such as protein expression (e.g., using immunofluorescence), morphological characteristics (e.g., by analysis of nuclear staining as indicated by Giemsa staining) and molecular characteristics (e.g., genomic analysis).

All documents cited above are incorporated herein by reference.

1. A method of determining the proliferative potential of a cell comprising the steps of allowing the cell to adhere to a microtrench that restricts movement of the cell such that on cell proliferation one or more daughter cells are sequentially arranged in a line, observing the microtrench on at least two occasions and determining whether a cell has undergone proliferation.

2. A method of determining the effect on cells of a given cell type of one or more test agents comprising the steps of allowing a first cell to adhere to a first microtrench, allowing a second cell to adhere to a second microtrench, exposing the first cell to the one or more test agents and determining whether the first and second cells exhibit different behaviours to one another.

3. A method of selecting a cell which exhibits minimum clonogenic potential (MCP) comprising placing a first cell in a constrained environment such that, if the first cell undergoes cell cycle division, substantially all daughter cells are arranged sequentially in a line, identifying a daughter cell that undergoes a further cell cycle division as exhibiting MCP.

4. The method of claim 3, wherein the constrained environment is provided by a microtrench.

5. The method of any one of claims 1, 2 and 4, wherein the ratio of the width of the trench to the maximum length of the short axis of the cell type is about 2 or less.

6. The method of any one of claims 1, 2 and 4, wherein the microtrench is formed as a depression in the surface, each side of the trench being formed by a wall.

7. The method of any one of claims 1, 2 and 4, wherein the microtrench is defined by two or more walls projecting from the surface.

8. The method of any one of claims 6 and 7, wherein each wall has a wall top surface formed so as to discourage cell adhesion to the wall top surface.

9. The method of claim 8, wherein the wall top surface is 1-30 μm wide.

10. The method of claim 8 or claim 9 wherein the material forming the wall top surface comprises and/or is coated with a compound which discourages cell adhesion to the surface.

11. The method of any one of the preceding claims wherein the microtrench has a depth which prevents motility-based emigration of each one or more cell from the trench.

12. The method according to any preceding claim wherein the microtrench has a depth sufficient to prevent a cell which is attached but in motile interphase from passing another cell which is attached but in motile interphase.

13. The method of claim 11 or claim 12, wherein the microtrench has a depth of 1-5 μm.

14. The method of any preceding claim wherein an interior surface of the microtrench comprises and/or is coated with a compound which encourages cell adhesion.

15. The method according to any preceding claim wherein an interior surface of the microtrench comprises and/or is coated with a compound which alters the behaviour of one or more of each one or more cells.

16. The method according to any of claims 8 and 9 wherein the spacing between each wall top surface is such that cell entry by motility-based immigration is prevented.

17. The method according to any preceding claim wherein each one or more ends of the trench are formed such that cell entry by motility-based immigration is prevented.

18. The method according to any preceding claim, wherein the microtrench is less than 550 μm in length.

19. A cell culture microtrench being defined on or in a surface of a substrate, wherein the ratio of the width of the microtrench to the maximum length of the short axis of a cell type of interest is about 6 or less, the maximum length of the short axis of the cell type being measured when a cell is in attached or adherent mitotic states, and wherein the microtrench is less than 550 μm in length.

20. A cell culture microtrench being defined on or in a surface of a substrate, wherein the ratio of the cross-sectional area of the microtrench to the maximum cross-sectional area of a cell type of interest is about 2 or less, the cross-sectional area of the cell type being measured when a cell is in detached or suspended form, and wherein the microtrench is less than 550 μm in length.

21. An array comprising a plurality of microtrenches according to any one of claims 19 and 20.

22. An array according to claim 21 wherein the plurality of microtrenches are substantially parallel and each microtrench being separated by a wall having a wall top surface.

23. An array according to claim 22 wherein each wall top surface is arranged so as to prevent the movement of a cell from one wall top surface to another wall top surface.

24. An array according to claim 23 wherein the material forming each wall top surface is 1-30 μm wide and/or comprises and/or is coated with a compound which discourages cell adhesion to the surface.

25. An array according to any of claims 21-24 further comprising one or more markings which allows the user to determine the orientation of the array and/or the location within the array of each at least one microtrench.

26. A method according to claim 2 wherein at least one of the one or more test agents is applied to the surface of the first microtrench, or forms part of the material of the first microtrench, prior to the first cell adhering to the first microtrench.

27. A method according to claim 2 or 26 wherein at least one of the one or more test agents is introduced into the first microtrench after the first cell adheres to the microtrench.

28. A method according to any of claims 2, 26 and 27 wherein at least one of the one or more test agents is introduced into the interior of the first cell, either before or after the cell adheres to the first microtrench.

29. The method according to any one of claims 1 to 3, wherein the one or more cells is a tumour cell.

30. A method of transferring one or more cells contained in a microtrench to a different surface, comprising: bringing the microtrench and the surface into contact so that the one or more cell are in contact with the surface for a sufficient time to allow transfer of the cells to the surface, and removing the surface with the cells attached.

31. The method of claim 30, wherein the surface is a poly-L-lysine surface.
